(19) United States
(12) Patent Application Publication
(10) Pub. No.: US 2021/0057112 A1
(43) Pub. Date: Feb. 25, 2021

(54) METHOD AND SYSTEM FOR MOBILE TRIAGE

(71) Applicant: Viz.ai Inc., San Francisco, CA (US)

(72) Inventors: Christopher Mansi, San Francisco, CA (US); David Golan, San Francisco, CA (US)

(21) Appl. No.: 17/001,218

(22) Filed: Aug. 24, 2020

Related U.S. Application Data
(60) Provisional application No. 62/891,109, filed on Aug. 23, 2019.

(51) Int. Cl. G16H 80/00; G16H 50/20; G16H 50/30; G16H 10/60; G06F 16/901

(52) U.S. Cl. CPC G16H 80/00; G16H 50/20; G06F 16/9017; G16H 10/60; G16H 50/30

(57) ABSTRACT

A system for mobile triage includes a client application executing on a user device, and optionally includes and/or interface with any or all of: the user device and/or set of user devices; a computing system including one or more computing subsystems and/or remote computing subsystems; a lookup table; and/or any other suitable components. A method for mobile triage includes assessing a patient and triaging the patient based on the assessment. Additionally or alternatively, the method can include any or all of: initiating an assessment, determining a patient condition, collecting and integrating data, and/or any other suitable processes.

### CROSS-REFERENCE TO RELATED APPLICATIONS

This application claims the benefit of U.S. Provisional Application No. 62/891,109, filed 23 Aug. 2019, which is incorporated in its entirety by this reference.

### TECHNICAL FIELD

This invention relates generally to the medical diagnostic field, and more specifically to a new and useful system and method for mobile triage in the medical diagnostic field.

### BACKGROUND

In current triaging workflows, especially those in an emergency setting, a patient presents at a first point of care at which a set of diagnostic assessments (e.g., imaging, testing, consultation with an emergency room physician, etc.) are performed. At this point, it might be determined that the patient needs to go to a 2^{nd} point of care (e.g., a specialty center, hub, etc.) for treatment. For patients presenting with stroke symptoms, for instance, the patient is typically taken to a spoke facility (e.g., general hospital, urgent care, closest emergency room, etc.), where it is determined that the patient needs to be transferred to a hub (e.g., comprehensive stroke center) to receive appropriate treatment. The time that it takes to perform this assessment and then perform/initiate a transfer (instead of the patient being taken to the appropriate treatment facility in the first place) can result in any or all of: a significant amount of brain being compromised, a number of potentially viable treatment options decreasing, and/or severe patient harm or fatality.

Thus, there is a need in the triaging field to create an improved and useful system and method for decreasing the time it takes to determine and/or transport a patient to an appropriate first point-of-care.

### BRIEF DESCRIPTION OF THE FIGURES

FIG. 1 is a schematic of a system for mobile triage.

FIG. 2 is a schematic of a method for mobile triage.

FIGS. 3A-3S depict a variation of a method for mobile triage.

FIGS. 4A-4C depict a variation of an assessment of arm drift in a patient.

FIGS. 5A-5C depict a variation of an assessment of facial droop in a patient.

FIG. 6 depicts a variation of communication between users the method 200.

FIG. 7 depicts a schematic variation of the method 200.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

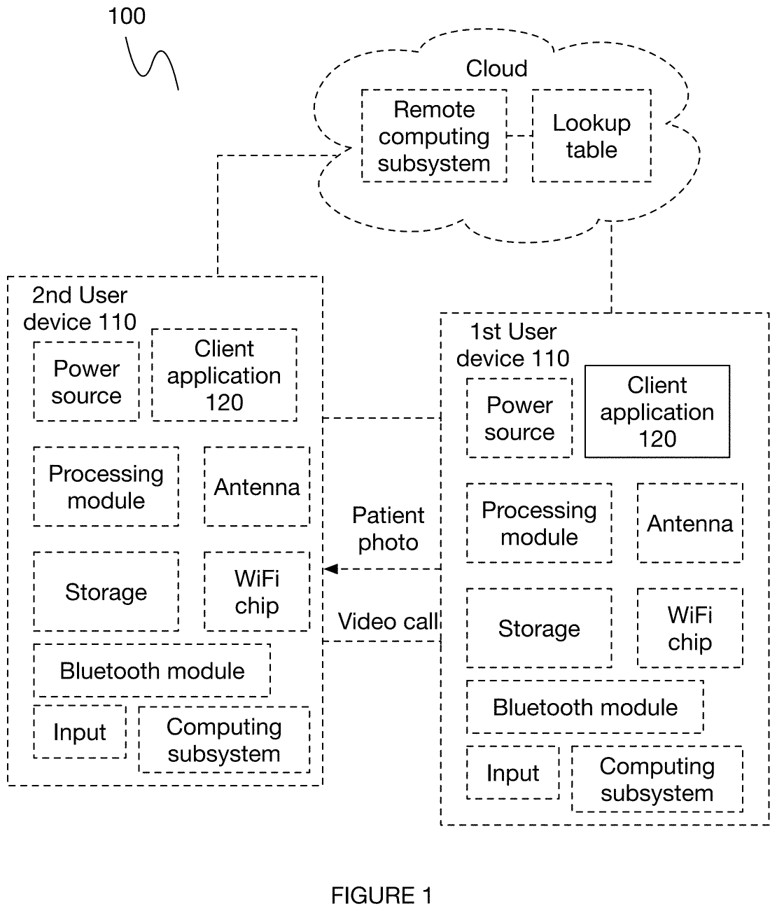

As shown in FIG. 1, a system 100 for mobile triage includes a client application 120 executing on a user device. Additionally or alternatively, the system 100 can include and/or interface with any or all of: the user device 110 and/or set of user devices; a computing system including one or more computing subsystems and/or remote computing subsystems; a lookup table; and/or any other suitable components. Further additionally or alternatively, the system can include or all of the systems, components, embodiments, and/or examples as described in any or all of: U.S. application Ser. No. 16/012,458, filed 19 Jun. 2018; U.S. application Ser. No. 16/012,495, filed 19 Jun. 2018; U.S. application Ser. No. 16/688,721, filed 19 Nov. 2019; and U.S. application Ser. No. 16/913,754, filed 26 Jun. 2020, each of which is incorporated in its entirety by this reference.

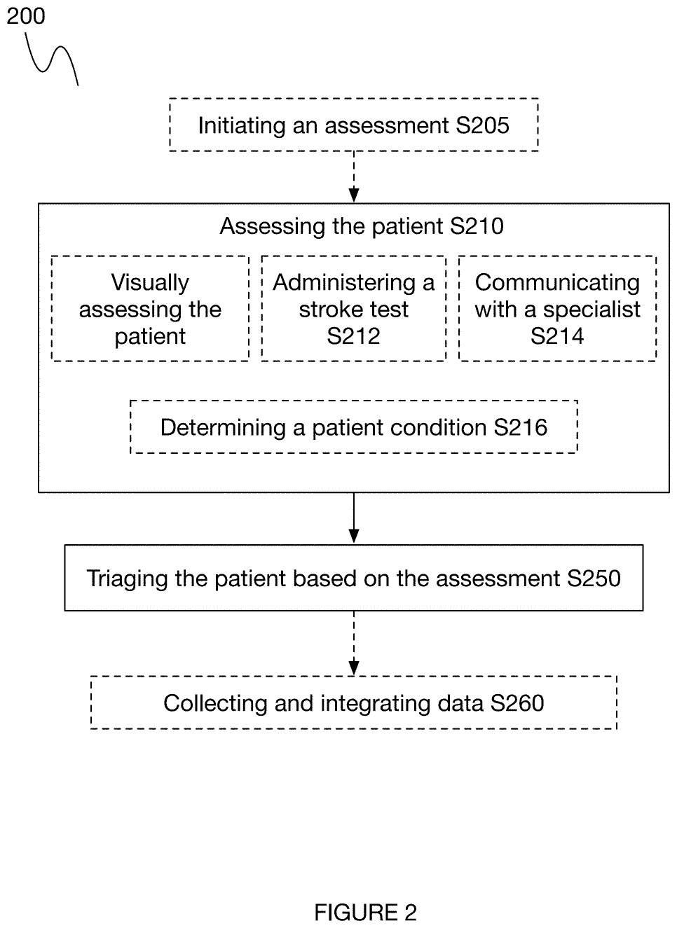

As shown in FIG. 2, a method 200 for mobile triage includes assessing a patient S210 and triaging the patient based on the assessment S250. Additionally or alternatively, the method 200 can include any or all of: initiating an assessment S205, determining a patient condition S216, collecting and integrating data S260, and/or any other suitable processes. Further additionally or alternatively, the method can include or all of the methods, processes, embodiments, and/or examples as described in any or all of: U.S. application Ser. No. 16/012,458, filed 19 Jun. 2018; U.S. application Ser. No. 16/012,495, filed 19 Jun. 2018; U.S. application Ser. No. 16/688,721, filed 19 Nov. 2019; and U.S. application Ser. No. 16/913,754, filed 26 Jun. 2020, each of which is incorporated in its entirety by this reference.

### 2. Benefits

The system and method for mobile triage can confer several benefits over current systems and methods.

In a first variation, the method and/or system confer the benefit of transporting the patient to an optimal point of care (e.g., hub, stroke center, etc.) as the first destination. In a specific example, the method enables the patient to be transported via an ambulance from the patient's initial site (e.g., home) to a stroke center without first going to a default emergency room and then being transferred to a stroke center from there.

In a second variation, the system and/or method confer the benefit of reducing (and/or eliminating) a number of transfers required to reach a particular point of care (e.g., final point of care, critical point of care, etc.).

In a third variation, the system and/or method confer the benefit of reducing a time required to make a decision of where to take a patient (e.g., hub, spoke, etc.). Additionally or alternatively, the system and/or method can confer the benefit of reducing a time until the performance of a particular procedure (e.g., first scan, surgery, administration of medication, etc.).

In a fourth variation, the system and/or method confer the benefit of increasing a number of opinions involved in the decision-making process of how to triage the patient (e.g., where to take patient, what procedures to perform, etc.). Additionally or alternatively, the system and/or method can increase a level of expertise of the opinion(s) involved in triaging the patient. In a specific example of this variation, the opinion of a specialist (e.g., a neurosurgeon, neurologist, etc.) is brought in to supplement and/or verify the assessment of a first responder (e.g., emergency medical technician [EMT], EMT-intermediate, emergency medical services [EMS] responder, paramedic, etc.) in determining where to take the patient.

In a fifth variation, the system and/or method confer the benefit of equipping emergency personnel (e.g., mobile emergency personnel, personnel working in an emergency room, etc.) with new and effective tools to assess the patient's condition and optionally a severity of the condition earlier than conventionally occurs in a triaging workflow (e.g., prior to arrival at a 1^{st} healthcare facility, prior to being seen by a specialist in an emergency room, etc.).

Additionally or alternatively, the method and system can confer any other suitable benefit(s).

### 3. System 100

As shown in FIG. 1, a system 100 for mobile triage includes a client application 120 executing on the user device. Additionally or alternatively, the system 100 can include and/or interface with any or all of: a user device 110 and/or set of user devices; a computing system including one or more computing subsystems and/or remote computing subsystems; a lookup table; and/or any other suitable components. Further additionally or alternatively, the system can include or all of the systems, components, embodiments, and/or examples as described in any or all of: U.S. application Ser. No. 16/012,458, filed 19 Jun. 2018; U.S. application Ser. No. 16/012,495, filed 19 Jun. 2018; U.S. application Ser. No. 16/688,721, filed 19 Nov. 2019; and U.S. application Ser. No. 16/913,754, filed 26 Jun. 2020, each of which is incorporated in its entirety by this reference.

The system preferably interfaces with one or more points of care (e.g., 1^{st} point of care, 2^{nd} point of care, 3^{rd} point of care, etc.), which are each typically a healthcare facility. A 1^{st} point of care herein refers to the healthcare facility at which a patient presents, typically where the patient first presents (e.g., in an emergency setting). Conventionally, healthcare facilities include spoke facilities, which are often general (e.g., non-specialist, emergency, etc.) facilities, and hub (e.g., specialist) facilities, which can be equipped or better equipped (e.g., in comparison to spoke facilities) for certain procedures (e.g., mechanical thrombectomy), conditions (e.g., stroke), or patients (e.g., high risk). Some variations of the system and method function to prevent the need for a 2^{nd} point of care (e.g., by transporting a patient to a hub as the 1st point of care). Additionally or alternatively, the system can interface with any suitable facilities based on any suitable data (e.g., when it is evident what condition a patient's symptoms reflect, when a patient has a prior history of a serious condition, when a patient condition has progressed to a high severity, when a hub facility is closest, randomly, etc.). A healthcare facility can include any or all of: a hospital, clinic, ambulance, doctor's office, imaging center, laboratory, primary stroke center (PSC), comprehensive stroke center (CSC), stroke ready center, interventional ready center, neurointerventional center, neurology clinic, or any other suitable facility involved in patient care and/or diagnostic testing.

A patient can be presenting with symptoms of a condition, no symptoms (e.g., presenting for routine testing), or for any can be presenting in any other way and with any suspected condition. In some variations, the patient is presenting with one or more stroke symptoms (e.g., hemorrhagic stroke symptoms, ischemic stroke symptoms, etc.), such as, but not limited to: weakness, numbness, speech abnormalities, facial drooping, headache, an inability to hold out his or her arms in a level orientation, difficulties with memory (e.g., short term memory, long term memory, etc.), less than full consciousness (e.g., partial consciousness, unconsciousness, etc.), and/or any other stroke symptoms. Additionally or alternatively, the patient can be presenting with other symptoms, such as those potentially related to another brain condition (e.g., aneurysm), conditions independent of a brain condition (e.g., cardiac condition, lung condition, etc.), and/or any other condition(s).

In variations where the patient is presenting with one or more symptoms, the symptoms can be any or all of: determined and/or detected by an emergency responder who subsequently initiates one or more processes of the method 200; detected by a remote user (e.g., specialist), such as through an initial teleconference; received from and/or determined based on a call with a 911 operator (e.g., call between patient or person calling on behalf of the patient and the 911 operator); and/or otherwise determined or received.

### 3.1 System: User Device 110

The system interfaces with and/or includes a set of one or more user devices no, each of which functions to support one or more client applications (e.g., as described below). Additionally or alternatively, the user device 110 can function to collect patient data (e.g., through one or more supplementary sensors of the user device), store patient data, route an emergency vehicle (e.g., along an optimal route, with a navigation application, etc.), communicate with a remote computing system and/or database (e.g., to pull patient information from an electronic health record [EHR] and/or an electrical medical record [EMR] database, to transmit information to a database, etc.), perform any or all of the processing required for the method 200, and/or perform any other suitable function(s).

The user device 110 is preferably a mobile device, but can additionally or alternatively be a stationary device, a device fixed within a vehicle (e.g., in an ambulance), or any other suitable device or combination of devices. Examples of the user device include a mobile phone (e.g., smartphone), tablet, watch, wearable device (e.g., glasses), computer (e.g., desktop computer, laptop, etc.), workstation (e.g., clinical workstation at a healthcare facility), or any other suitable user device. The user device can include power storage (e.g., a battery), processing systems (e.g., CPU, GPU, memory, etc.), user outputs (e.g., display, speaker, vibration mechanism, etc.), user inputs (e.g., a keyboard, touchscreen, microphone, etc.), a location system (e.g., a GPS system), sensors (e.g., optical sensors, such as light sensors and cameras, orientation sensors, such as accelerometers, gyroscopes, and altimeters, audio sensors, such as microphones, etc.), data communication system (e.g., a WiFi module, BLE, cellular module, etc.), or any other suitable component.

The user device no is preferably associated with (e.g., accessible by, on the person of, owned by, provided to and/or lent to through an employment agreement, etc.) an emergency responder (e.g., EMT, EMT-intermediate, EMS, paramedic, first responder, police officer, fireman, etc.), such that the emergency responder can assess one or more patients.

The system 100 further preferably interfaces with and/or includes one or more user devices 110 associated with any or all of: a specialist (e.g., neurospecialist, neurosurgeon, neuroradiologist, neurointerventionalist, neurologist, etc.) and/or multiple specialists (e.g., a first specialist, other healthcare worker, the patient, a user associated with the patient (e.g., family member present in an ambulance with the patient, user present at the patient pick-up site, etc.), a driver (e.g., ambulance driver, driver transporting the patient to a point of care, etc.), or any other suitable user.

In preferred variations, the system interfaces with and/or includes at least a first user device associated with an emergency responder (e.g., EMT) and a second user device associated with a specialist and/or other user (e.g., tele-doctor) with expertise to help assess the patient, wherein the first user device and the second user device are configured to communicate with each other in the assessment of a patient. In specific examples, communication between the first user device and the second user device is initiated upon selection of the physician at a client application executing on the first user device by the emergency responder. Additionally or alternatively, the physician can be any or all of: automatically selected and optionally automatically contacted; selected at and/or by a remote computing system (e.g., with one or more algorithms, with a machine learning process, etc.); self-selected (e.g., volunteering participation); and/or otherwise selected.

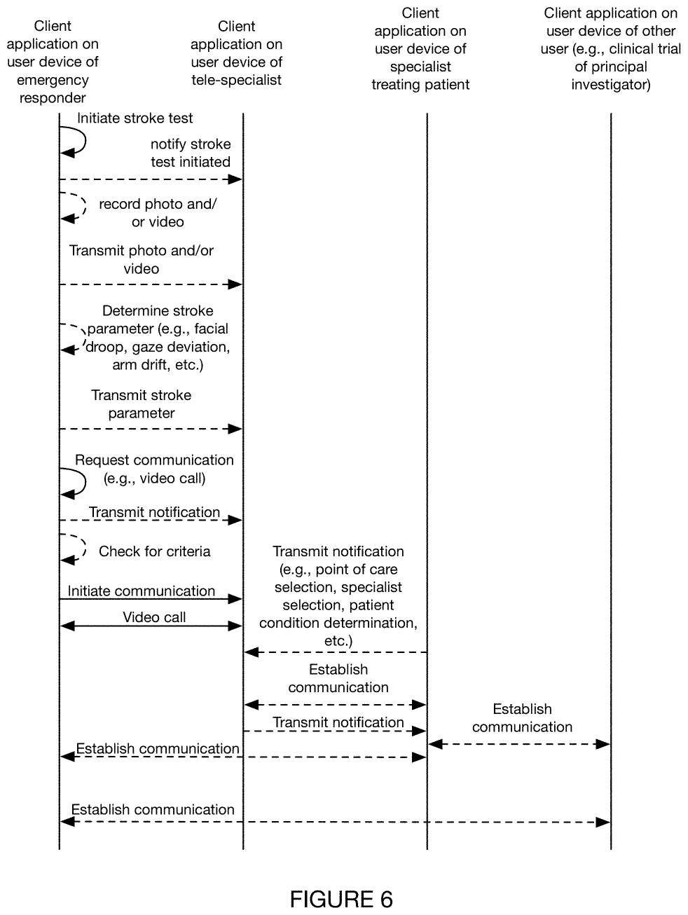

Additionally or alternatively, the system can interface with and/or include any other user devices (e.g., as shown in FIG. 6), such as user devices associated with any other users involved in any or all of: the treatment of the patient (e.g., care team members at the healthcare facility to which the patient is being transported, administrative workers such as those organizing treatment of the patient, etc.); enrollment of the patient in a clinical trial (e.g., principal investigator of a clinical trial, research coordinator at and/or associated with the healthcare facility to which the patient is being transported, etc.); transportation of the patient to a healthcare facility (e.g., ambulance driver, any emergency responder onboard or offboard an ambulance, etc.); users at a spoke facility to which the patient is initially transported to; users at a hub facility to which the patient is being transported to; the patient; the patient's family; and/or any other suitable users. The user devices are preferably in communication (e.g., selective communication) through instances of a client application (e.g., as described below) executing on each of the user devices, but can additionally or alternatively be otherwise connected and/or in communication.

In some variations, for instance, in addition to the user device associated with the emergency responder, the set of user devices includes user devices associated with multiple specialists, such as a user device associated with a first specialist involved in the assessment of the patient and a user device associated with a second specialist involved in the treatment of the patient. The second specialist can be in communication with (e.g., through one or more client applications such as those described below) any or all of: the first specialist (e.g., to be informed by the first specialist of the state of the patient, a suspected condition of the patient, a potential treatment option for the patient, etc.); the emergency responder and/or other individuals traveling with the patient (e.g., to receive updated information from the emergency responder such as estimated time of arrival, to inform the emergency responder of where to take the patient, etc.); the patient; other members of the patient's care team (e.g., to begin coordinating treatment); a clinical trial researcher and/or coordinator (e.g., to initiate enrollment of the patient in a clinical trial, etc.); and/or any other suitable users and associated user devices.

### 3.2 System: Client Application 120

The system 100 includes a client application 120 configured to execute on one or more of the user devices 110. Additionally or alternatively, the system can include multiple client applications 120 (e.g., a different client application for each user), a client application 120 configured to operate on a different device, or any other suitable client application.

The client application 120 executing on the user devices of emergency responders preferably functions to provide the framework (e.g., questionnaires, stroke tests, instructions, etc.) with which an emergency responder can assess a patient suspected of presenting with a stroke.

As such, the client application 120 preferably includes one or more inputs (e.g., buttons on a touch-sensitive screen of a user device) configured to receive a selection and/or information from a user (e.g., emergency responder, patient, specialist, etc.). The client application can include a set of inputs, for instance, used in creating a patient profile (e.g., used in the selection of “add patient,” “create new patient,” “assess patient,” etc.). This can be used in collecting any or all of the following information from a patient: patient name, patient healthcare facility (e.g., preferred facility, previously-visited facility, predicted facility, recommended facility, facility of primary care doctor and/or specialist, etc.), patient medical record number (MRN), gender, date of birth, other demographic information (e.g., age, sex, height, weight, etc.), medical information and/or historical information (e.g., disease state history, pre-existing conditions, stroke history, comorbidities, medications being taken by the patient, etc.), and/or any other suitable information.

The client application can additionally or alternatively include a set of inputs and/or outputs for requesting a call (e.g., used in a “request call” option for an emergency responder) with another user, preferably a specialist, but additionally or alternatively another healthcare worker, another emergency responder, or any other suitable user. Requesting a call can optionally trigger one or more requests for additional information to be provided to the recipient (e.g., prior to the specialist joining a call, during a call, as a follow-up after a call, etc.), such as a request for any or all of: a photo of the patient (e.g., taken by an emergency responder, etc.), additional patient information (e.g., current status, emergency responder's current impression of patient state, etc.), stroke scale information (e.g., prompting an emergency responder to record a stroke scale), test results (e.g., prompting an emergency responder to perform one or more stroke tests, or any other requests for any suitable information. In some variations, for instance, a call (e.g., video call) or other communication is initiated in the method 200 in response to determining that any or all of the above has been recorded and/or entered at the client application of the emergency responder. In specific examples, a request (e.g., in the form of a notification at the client application, in the form of a text message, etc.) for a call and/or communication is made to the specialist (and/or any other suitable recipient), wherein the call and/or communication is only initiated once predetermined criteria (e.g., completion of a stroke test, entering of patient information, determination of a facial droop parameter, determination of a gaze deviation parameter, determination of an arm drift parameter, etc.). Additionally or alternatively, a notification to the recipient can be provided at any suitable time(s) during the method (e.g., in response to a stroke test being initiated, in response to a stroke score above a predetermined threshold being calculated, in response to a facial droop parameter above a predetermined threshold being calculated, in response to a gaze deviation parameter above a predetermined threshold being calculated, in response to an arm drift parameter above a predetermined threshold being calculated, etc.); a call can otherwise be initiated (e.g., in absence of a set of criteria, while the set of criteria are being determined, wherein the specialist on the call helps determine the set of criteria, etc.); and/or the client application can be otherwise configured.

Additionally or alternatively, the client application can receive (and optionally provide a prompt for) a photo of the patient, wherein a processing system (e.g., of the user device, of the client application, of a remote computing system, etc.) analyzes the photo (e.g., through one or more computer vision techniques, with one or more machine learning processes, etc.), to make an assessment of the patient. In some variations, for instance, one or more algorithms (e.g., for face detection, facial recognition, etc.) are used to analyze the photo and determine one or more features of the user's face (e.g., a facial asymmetry, a distance between two or more facial features and or facial markers, a drooping of the left side of the face, a drooping of the right side of the face, an asymmetry, etc.), which can be used to identify one or more stroke indicators. In additional or alternative variations, one or more algorithms and/or machine learning processes can be implemented to analyze the photo and determine features of any or all of: the user's eyes (e.g., gaze deviation); the user's body (e.g., arm drift, gate abnormalities, etc.); and/or any other feature.

In addition or alternative to a photo, a video of the patient can be recorded to perform any or all of these assessments (e.g., facial droop, gaze deviation, arm drift, etc.).

The client application can optionally provide one or more outputs, such as a set of instructions for an emergency responder, which can be used for any or all of: checking for signs of stroke, treating the patient, preventing further damage to the patient, providing driving instructions for where to transport the patient, the location of a selected point of care, which specialist to contact, or any other suitable instructions.

Additionally or alternatively, the client application can trigger (e.g., call on) and/or prompt a secondary application of the user device to receive, process, and/or provide any suitable information. The secondary application can include a native application (e.g., native messaging application, native phone call application, native navigation application) on the user device and/or any other suitable application. In some variations, for instance, the client application triggers navigation instructions to be updated at a secondary application of the user device in response to a point of care to which the patient is being transported is updated (e.g., upon consulting with a specialist).

The client application can further optionally be used to calculate and/or assign one or more scores to a patient (e.g., at a computing subsystem of the user device, at a remote computing subsystem, etc.), such as a patient score associated with one or more stroke assessments. The score can be calculated and/or stored in response to any suitable trigger, such as any or all of: the completion of an assessment, a predetermined amount of time has passed without reaching out to a specialist (e.g., for a video call), upon detection of a stroke indicator/symptom, upon calculating that a stroke likelihood is above a predetermined percentage likelihood, or based on any other suitable trigger. The score can be immediately communicated to a specialist, stored at the client application, transmitted to and stored at a patient database (e.g., EMR, EHR, cloud storage, etc.), provided to the emergency responder, or otherwise used.

The client application preferably provides one or more visuals (e.g., automatically, upon request by a user, based on a time, based on a location in relation to a point of care, etc.), which can function to convey information related to any or all of: a point of care location (e.g., indicated on a map view, updated in real or near real time with updated location/proximity, including an estimated time of arrival, indicating an optimal part of the facility to take the patient, etc.), a point of care parameter (e.g., number of beds available, treatment options available, capabilities, etc.), a specialist parameter (e.g., specialist availability, number of specialists on call, which particular specialists are on call, specialist schedule, time that a specialist was last active, time that a specialist was last logged into a client application, specialist availability to be on a video call, specialist rating, etc.), or any other suitable parameter or other information.

The system 100 can additionally or alternatively include a client application 120 configured for a specialist and/or any other user to establish communication and share information with the emergency responder. The client application for the specialist can be any or all of: partially or fully identical to the emergency responder's client application (e.g., able to perform stroke tests, able to determine parameters such as facial droop and/or gaze deviation and/or arm drift, etc.); partially or different from the emergency responder's client application (e.g., able to access additional information such as additional databases, unable to perform mobile stroke tests, etc.); any combination; and/or otherwise configured.

Any or all of the client applications can additionally or alternatively be configured based on any or all of: personal preferences; professional level and/or extent of granted access; healthcare facility preferences; and/or any other parameters.

The system 100 can further additionally or alternatively include a client application specific to any or all of: a research coordinator (e.g., principal investigator, research, administrator, etc.) associated with a clinical trial (e.g., configured to transmit a consent form for the clinical trial to the patient and/or the emergency responder and/or the specialist; configured to initiate the checking of a set of inclusion criteria based on any or all of the patient information and inputs; etc.); a family member of the patient; the patient; an administrative assistant and/or any healthcare worker; an ambulance driver; and/or any other suitable users.

In variations of the system having multiple users interacting with a set of client applications, the client applications can be different among users (e.g., same for each type of user group, customized to preferences of each user, etc.). In some examples, for instance, the system can include a client application configured for an emergency responder (e.g., including transportation/traffic information, include stroke test information, etc.) and a client application configured for a specialist (e.g., optimized for video calls, displaying patient information, integrating with an EMR database, etc.). Alternatively, a single client application can be used by multiple and/or all types of users.

In a first variation of the system 100, the system includes a first version of the client application for emergency responders and a second version of the client application for specialists, wherein communication is established between the client applications in response to the emergency responder requesting communication with the specialist and the specialist accepting, and optionally in response to a set of criteria first being satisfied.

### 3.3 System: Computing System

The system can optionally include a computing system (equivalently referred to as a processing module), which can function to perform any or all of the computing and/or processing performed in the method 200, such as that used to: assess the patient; determine a point of care; determine a set of specialist options and/or select a specialist; determine a patient condition; process images and/or video taken of the patient; calculate a score (e.g., a stroke test score); determine and/or update navigation instructions for an ambulance; and/or determine any other suitable information or perform any other suitable actions.

The computing system can be part of and/or distributed among any or all of: a set of one or more user devices, a remote computing system (e.g., cloud computing system), a computing system associated with the specialist and/or a healthcare facility of the specialist (e.g., a workstation), a computing system associated with any healthcare facility (e.g., workstation at a 1^{st} point of care, workstation at an emergency room, etc.), or any other suitable computing system.

The computing system preferably includes and/or interfaces with memory and/or storage, which can be used to store, but is not limited to storing, any or all of: a set of lookup tables, which can function to reference a specialist and/or healthcare facility (e.g., based on ambulance location, based on suspected patient condition, based on recommended treatment option, based on stroke test score, based on specialist recommendation, based on home facility of patient, etc.); one or more databases (e.g., including historical patient information, an EMR and/or EHR database, etc.); and/or any other suitable information.

The computing system is preferably configured to implement and/or store a set of algorithms and/or machine learning models (e.g., deep learning models, neural nets/neural networks, etc.), wherein the set of algorithms and/or machine learning models can be configured to determine (e.g., compute, calculate, etc.) any or all of: a stroke test score; facial droop parameter (e.g., distance of droop, side of droop, etc.); gaze deviation parameter (e.g., angle of gaze deviation, eye experiencing gaze deviation, etc.); limb drift parameter (e.g., distance of drift, limb of drift, arm of drift, etc.); aggregated stroke parameter; suspected brain condition (e.g., stroke, hemorrhagic stroke, ischemic stroke, etc.); and/or any other suitable parameters.

In a set of preferred variations, the system 100 includes a computing system distributed among a remote computing subsystem (e.g., cloud-based computing system) and a set of one or more computing subsystems onboard one or more user devices, wherein any or all of the computing subsystems are involved in the processing of a stroke test and/or visual data of the patient (e.g., video taken of the patient, photos taken of the patient, etc.). Additionally or alternatively, any or all of the computing system can be involved in the selection of one or specialists, the selection of one or more healthcare facilities, and/or any other suitable processing.

### 3.4 System: Variations

In a first variation, the system includes a client application executing on a user device (e.g., a mobile phone) of an emergency responder and a client application executing on a user device (e.g., a mobile phone, workstation, etc.) of a specialist, wherein at least the client application of the emergency responder is configured to assess the patient through one or more stroke tests as well as initiate a video call with a specialist. The client application of the emergency responder is further preferably configured to record a photo and/or video of the patient, which is processed to determine (e.g., with a set of algorithms, with a set of deep learning models at a remote computing system, etc.) one or more stroke parameters, such as any or all of: a facial droop parameter, a gaze deviation parameter, a limb drift parameter, and/or any other suitable parameters.

In a first set of specific examples, communication (e.g., messaging, video conferencing, etc.), such as in the form of a tele-consultation is selectively enabled (e.g., in response to a call being requested after a stroke test is performed). Additionally or alternatively, communication can be constantly enabled.

### 4. Method

As shown in FIG. 2, method 200 for mobile triage includes assessing a patient S210 and triaging the patient based on the assessment S250. Additionally, the method 200 can include any or all of: initiating an assessment S205, determining a patient condition S216, collecting and integrating data S260, and/or any or all of the methods, processes, embodiments, and/or examples as described in any or all of: U.S. application Ser. No. 16/012,458, filed 19 Jun. 2018; U.S. application Ser. No. 16/012,495, filed 19 Jun. 2018; U.S. application Ser. No. 16/688,721, filed 19 Nov. 2019; and U.S. application Ser. No. 16/913,754, filed 26 Jun. 2020, each of which is incorporated in its entirety by this reference, and/or any other suitable processes performed in any suitable order. The method 200 can be performed with a system 100 as described above and/or any other suitable system.

The method 200 functions to decrease the time required to transfer a patient having a suspected time-sensitive condition (e.g., brain condition, stroke, hemorrhagic stroke, ischemic stroke, large vessel occlusion (LVO), cardiac event, trauma, etc.) to a healthcare facility equipped to treat the patient. As such, the method 200 can function to enable any or all of: early stage triaging of a patient (e.g., performing a stroke assessment prior to being placed in an ambulance), mobile triaging of a patient (e.g., video calling a specialist while patient is in an ambulance, dynamically routing a patient to a point of care based on patient development, etc.), or any other suitable decision making.

The method 200 is preferably performed in response to a patient or a person associated with the patient interacting with an emergency responder (e.g., EMT, paramedic, a family member or friend, etc.), such as upon arrival of an ambulance to a location of the patient. The method 200 can be initiated automatically (e.g., with the receipt of a 911 call, with the read receipt of a notification to a client application of the emergency responder, with the read receipt of a notification to a client application of a specialist, etc.), and/or in response to any or all of the following triggers: an emergency responder reaching the patient (e.g., emergency responder arrives on scene, patient is placed in an ambulance, etc.), a patient presenting an indicator associated with stroke (e.g., prior to ambulance reaching patient, upon ambulance reaching patient, after ambulance reaches patient, while patient is in ambulance, etc.), while a patient is in transit to a point of care (e.g., between patient's location at the time of a 911 call and a first point of care, between a first point of care and a second point of care, etc.), after a patient symptom (e.g., headache) persists longer than a predetermined threshold of time, in response to review of the patient by a specialist (e.g., through a video call as described below, based on a photo and/or other information sent to the specialist, etc.), and/or or based on any other suitable trigger.

The method can be performed, in part or in whole, by any or all of the following users: an emergency responder, specialist (e.g., surgeon) or other healthcare worker (e.g., physician, ER doctor, administrative worker, technician, nurse, etc.), patient (e.g., patient answering one or more stroke test questions on an EMT's mobile device), a user associated with the patient (e.g., who can function to initiate the method prior to an EMT arriving, who can perform part of the method while an EMT is occupied, etc.), a driver (e.g., of an ambulance), pilot, or any other suitable user. The method is preferably additionally performed, at least in part, with a computing system as described above, but can additionally or alternatively be performed with any other suitable components and/or combination of components.

Part or all of the method 200 can be integrated into (e.g., supplement, reduce a time required for, etc.) and/or replace any or all of the current/conventional protocols of an emergency responder. In some variations, for instance, the method 200 functions to prompt and log the results of a stroke assessment performed by an emergency responder upon reaching the patient. The stroke assessment is conventionally a stationary stroke assessment performed outside of and prior to the patient's entry into the ambulance; in preferred variations, the method 200 enables this assessment to optionally be performed in a mobile setting (e.g., while the patient is onboard the ambulance), which can confer the benefit of reducing the time to treatment. Additionally or alternatively, part or all of the method can be performed in any or all of the following ways: in parallel with a current protocol of an emergency responder (e.g., to gather more information which can be provided to a physician when the patient reaches the first point of care, to gather information in order to determine a condition of a patient, etc.); in addition to a current protocol of an emergency responder (e.g., enable a video call with a physician); in a way that enables the emergency responder to not have to perform part or all of an existing protocol (e.g., a stroke test can be assessed and logged automatically without user intervention); in a way that enables an emergency responder protocol to be performed in any other suitable way; and/or in any other suitable way(s).

Additionally or alternatively, part or all of the method 200 can modify, adjust, supplement, remove requirements from, or otherwise affect one or more current protocols performed by a specialist. The specialist preferably refers to a physician (e.g., neurospecialist, neurosurgeon, neurointerventionalist, surgeon, neurologist, primary care physician, specialist who ultimately interacts with and/or treats the patient, specialist at the point of care to which the patient is transported, specialist who does not treat the patient, specialist who assesses the patient, member of the patient's eventual care team, etc.), but can additionally or alternatively refer to and/or include a person associated with a clinical trial, a non-physician, and/or any other suitable individual(s). In some variations, for instance, the method 200 can enable a specialist to do any or all of: assess (e.g., see, talk to, etc.), triage, and/or receive the results of an assessment of a patient prior to when a specialist (e.g., the same specialist, a different specialist, etc.) would normally see the patient (e.g., upon arrival to the specialist facility); minimize and/or eliminate a stroke assessment (e.g., series of stroke tests) performed when the patient reaches the specialist's point of care (e.g., since one or more stroke tests were performed in transit to the point of care); enable a specialist to treat the patient in a reduced amount of time (e.g., based on the information gathered by the specialist during a video call); prevent the patient from reaching the specialist (e.g., if the specialist recommends that the patient instead be transported to another specialist or physician); enable a specialist to receive up-to-date (e.g., real time, substantially real time, during the time period of the ambulance ride, etc.) or have any other suitable outcome for the specialist.

The method 200 is preferably performed at one or more of: an initial site of the patient (e.g., location where ambulance meets patient, patient home, where patient experiences stroke symptoms, workplace, etc.), a point of care (e.g., while patient is in a waiting room, while patient is in an emergency room, while patient is waiting for care, etc.), a vehicle (e.g., ambulance, car, helicopter, etc.), or at any other suitable location.

### 4.1 Method—Initiating an Assessment S205

The method 200 can include initiating an assessment S205, which functions begin observation of a patient and/or the collection of data relevant to the patient condition. Additionally or alternatively, S250 can function to protect confidential patient information (e.g., by requiring verification of a user accessing a client application), or perform any other suitable function.

S205 can optionally include any or all of: verifying and/or validating an identity of one or more users (e.g., EMT, specialist, etc.) which can function to ensure that patient data is collected compliantly (e.g., by associating the data with a particular user, by not opening a client application until the user is verified, etc.), that data is collected from a proper user (e.g., patient), that recommendations and determinations are made using accessible and optimal data (e.g., EMR data, EHR data, home hospital of EMT drivers, current list of working specialists, etc.), and/or ensure any other suitable outcome. In some variations, S205 includes a login process for accessing a client application (e.g., the client application as described above, a secondary client application executing on the user device, etc.) for any or all of: the emergency responder (e.g., with a password, face recognition, fingerprint, thumbprint, etc.), the specialist, the patient, and/or any other suitable individual. Additionally or alternatively, a login process of the user device (e.g., face recognition, fingerprint, thumbprint, etc.) can be used to verify an identity of the user, another suitable verification process can be implemented, and/or the method can be performed in absence of the verification process.

S205 is preferably performed in response to an emergency responder reaching a patient (e.g., in the event that the emergency responder suspects a stroke condition, based on symptoms described in a 911 call, etc.) and/or when an emergency responder first notices a stroke symptom (e.g., when meeting the patient, when transporting the patient, etc.). Additionally or alternatively, S205 can be performed automatically, upon prompting by another user (e.g., specialist, patient, etc.), once the patient has been taken into the ambulance, prior to the patient being taken into the ambulance, in response to a diagnostic procedure (e.g., upon the generation of a set of diagnostic images at a first point of care), and/or at any other suitable time(s).

S205 can be performed a single time or multiple times throughout the method (e.g., sequentially, at different times in the method, once a patient condition has progressed, etc.).

In one set of variations, S205 is performed by an emergency responder (e.g., EMT) opening up (e.g., and logging into) a client application upon reaching a patient displaying at least one stroke symptom (e.g., during the time when an EMT would conventionally perform a stationary physical assessment of a patient).

In another set of variations, S205 is performed by an emergency responder in response to consulting with a specialist (e.g., in a teleconsultation video call, after sending a photo of the patient, after messaging the specialist, etc.), wherein the specialist recommends the assessment.

Additionally or alternatively, S205 can be performed in any suitable way and in response to any suitable triggers.

### 4.2 Method—Assessing the Patient S210

The method preferably includes assessing the patient S210, which functions to determine a point of care for the patient. Additionally or alternatively, S210 can function to determine a suspected condition of (e.g., diagnose) a patient, determine a recommended treatment option (e.g., mechanical thrombectomy, stent placement, the administration of a tissue plasminogen activator, etc.) for a patient, consult a specialist for an expert opinion and/or analysis of the patient, redirect a patient to an optimal point of care (e.g., stroke center) as opposed to an original destination (e.g., emergency room, spoke facility, etc.), or perform any other suitable function.

S210 is preferably performed in response to S205, but can additionally or alternatively be performed in absence in S205, automatically, or based on any of the possible set of triggers described above. S210 is preferably performed by an emergency responder and optionally a specialist (e.g., during an intra-ambulance teleconsult), but can additionally or alternatively be performed by any other suitable individuals and/or with any suitable computing system (e.g., computing subsystems described above). Additionally or alternatively, S210 can involve the patient (e.g., to self-assess, to provide answers and/or perform commands related to a stroke test, etc.), solely one or more specialists or other healthcare facility workers (e.g., if the patient has already reached a first point of care), or any other suitable user.

S210 can be performed while the patient is at a stationary location (e.g., pickup point, first point of care, etc.), at a mobile location (e.g., in ambulance while in transit to a first point of care), or any combination of both.

Assessing the patient S210 preferably includes visually assessing the patient for one or more visually manifesting stroke symptoms, such as any or all of: facial droop, gaze deviation or other gaze irregularities, limb drift (e.g., arm drift), imbalance, or any other symptoms. Visually assessing the patient can be performed manually by an emergency responder (e.g., and logged into the client application); additionally or alternatively, visually assessing the patient can be performed, at least in part, using a camera (e.g., of a user device, installed in an ambulance, activated by an in communication with the client application, etc.) and optionally aa set of algorithms and.or machine learning models (e.g., at the computing system, at a computing subsystem of a user device, at a remote computing subsystem). One or more cameras can be used, for instance, to collect a photo or video which is subsequently automatically analyzed (e.g., with one or more computer vision techniques) at a computing system (e.g., computing subsystem of user device, remote computing system, etc.), manually analyzed (e.g., by a specialist), occurring as part of a video call with a specialist, or otherwise taken. A camera is preferably in communication with (e.g., initiated by, controlled by, etc.) a client application as described above, but can additionally or alternatively be in communication with another client application (e.g., GPS application, maps, etc.), any or all of the computing system, one or more databases (e.g., for storage at a remote server in communication with the remote computing system, for storage at an EMR and/or EHR database, etc.), and/or any other suitable components.

Assessing the patient S210 can additionally or alternatively include an auditory assessment of the patient, which can be used for assessing and/or monitoring patient speech (e.g., cadence, content, etc.), detecting and/or recording patient responses to prompted questions, recording a conversation with a patient, and/or performing any other assessment. The auditory assessment can optionally test a memory of the patient, such as through any or all of: a set of questions (e.g., what the patient's name is, what year it is, who the current president is, etc.) to which the patient provides answers; a phrase/sentence spoken to and/or played for the patient that the patient is asked to repeat; and/or through any other auditory assessment(s).

Additionally or alternatively, a patient can be assessed through any other types of assessment, such as, but not limited to: a written or drawn assessment (e.g., to see how a patient reproduces a particular drawn figure, to see how patient transcribes a passage spoken by another person, to see how a patient spaces words in a written passage, etc.), the measurement of one or more vital signs, or any other form of assessment.

In preferred variations, assessing the patient S210 includes administering one or more stroke assessments (e.g., stroke tests) S212, which can function to detect for one or more stroke indicators (e.g., stroke symptoms), determine a severity of one or more stroke indictors, calculate and/or assign a patient score (e.g., likelihood that patient is experiencing a stroke, severity of a suspected or confirmed stroke, etc.), or otherwise assess the patient. S212 can include any or all of the stroke tests described above; additionally or alternatively, S212 can include the administration of any other suitable stroke assessments. The stroke assessment can include and/or be determined based on a standard stroke scale such as, but not limited to, any or all of: the Cincinatti Prehospital Stroke Severity Scale [CP-SSS], the Los Angeles Motor Scale [LAMS], the National Institute of Health Stroke Scale [NIHSS], the Rapid Arterial oCclusion Evaluation [RACE], and/or any other suitable stroke scale. Additionally or alternatively, the stroke assessment can be otherwise determined and/or defined.

The stroke test can prescribe any or all of the assessments described above and/or below, and can be any or all of: based on a predetermined set of standard stroke tests (e.g., as described below); dynamically determined (e.g., adapted based on the results of each sub-assessment, terminated upon detection of a score or sub-score above a predetermined threshold, terminated upon detection of a score or sub-score below a predetermined threshold, etc.); selected from a set of options by an emergency responder and/or specialist; built from a set of assessment modules by an emergency responder and/or specialist; determined based on one or more machine learning processes; and/or otherwise determined.

S212 preferably includes one or more visual assessments of the patient, which can assess for any or all of: stroke indicators can be perceived by an untrained eye, stroke indicators that can be perceived by a trained eye, stroke indicators that are beyond or nearly beyond human perception (e.g., facial droop less than a predetermined minimum threshold, etc.), or any other indicators. Any or all of the visual assessments can be performed in and/or with prompting by the client application (e.g., as described above). Additionally or alternatively, the visual assessments can be otherwise performed.

The visual assessments can assess for facial droop, which can be determined and/or quantified based on any or all of the following parameters: a binary presence of droop, an amount (e.g., as measured in a maximum distance) of droop, a region of droop (e.g., which side of the patient's face, a location of maximum facial droop, a closest facial feature with relation to the facial droop, etc.), a progression of facial droop (e.g., rate at which a facial droop is worsening), or any other suitable parameter.

A facial droop parameter can be determined based on one or more automated image processing techniques, such as any or all of: computer vision techniques, algorithms, deep learning algorithms (e.g., neural nets, convolutional neural nets, etc.), and/or any other image processing techniques.

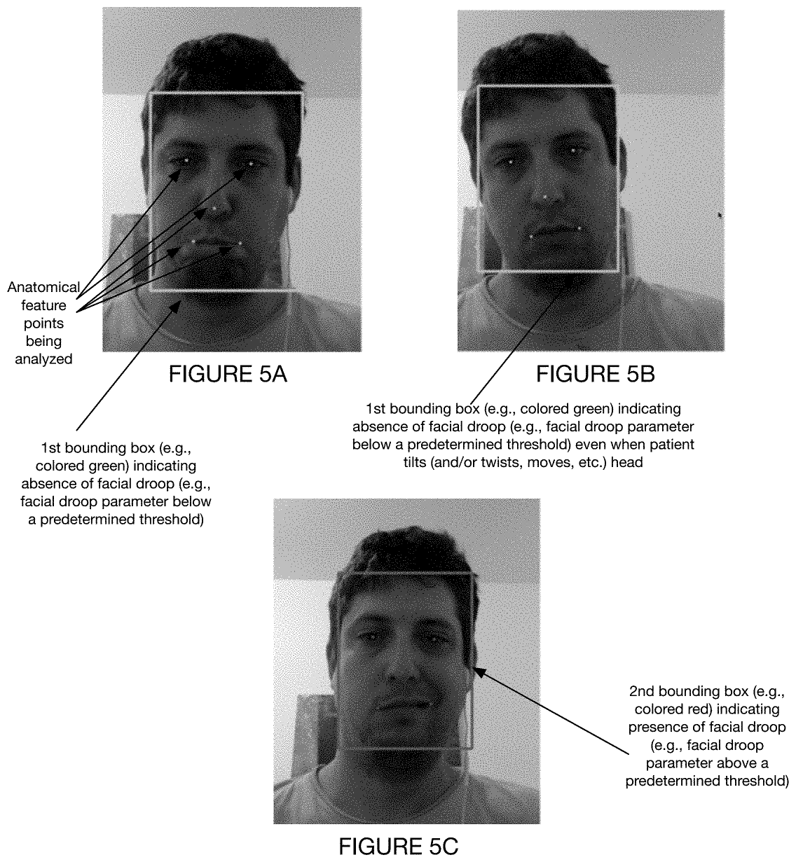

Visually assessing the patient preferably includes identifying a predetermined set of landmarks on the patient's face and determining a parameter (e.g., distance, angle, etc.) between any or all of the set of landmarks. By comparing the parameter with one or more thresholds, a determination of facial droop—and optionally a facial droop parameter—can be determined. The landmarks preferably correspond to anatomical features, such as any or all of: patients' eyes (e.g., pupil, corner of eye, tear duct, lid, etc.), nose (e.g., tip of noise, right nostril, left nostril, midpoint between nostrils, top of bridge, etc.), mouth (e.g., left corner, right corner, lower lip midpoint, upper lip midpoint, etc.), eyebrows, forehead, chin, ears, neck, or any other suitable feature. Additionally or alternatively, the features can include non-anatomical features (e.g., midpoints, endpoints, center point, intersection of bisecting axes, etc.), user-specific features (e.g., freckles, moles, scars, etc.), or any suitable features. In specific examples (e.g., as shown in FIGS. 5A-5C), the following points are identified and analyzed (e.g., distance between them calculated) to determine a presence of facial droop and optionally a parameter (e.g., length/distance of droop, percentage of droop, etc.) quantifying it: left eye pupil, right eye pupil, tip of noise, left corner of mouth, and right corner of mouth. Additionally or alternatively, any other suitable features can be used and analyzed.

In preferred variations, a set of trained neural nets (e.g., deep neural nets, convolutional neural nets, etc.) are used to identify (and optionally track in a video) the set of features. The set of neural nets are preferably finetuned based on images of stroke patients (e.g., to learn what facial droop looks like in stroke), but can additionally or alternatively be otherwise trained and/or finetuned. Additionally or alternatively, facial droop can be determined in absence of machine learning.

Comparing the parameter with a threshold can include any or all of: comparing one or more parameter values (e.g., distance, angle, etc.) with a predetermined anatomical threshold (e.g., aggregated anatomical value, average value, maximum anatomical value, minimum anatomical value, etc.), a corresponding value for an opposing side of the face (e.g., comparing a right side distance with a corresponding left side distance), or any other suitable value or threshold. In some examples, for instance, a parameter (e.g., distance between a left eye and a left corner of the mouth) is determined and compared with an anatomical threshold (e.g., average value, maximum value, maximum value once scaled to correspond to a size of the user's head, etc.), wherein if the parameter has a value greater than the anatomical threshold (e.g., at least 1% greater, at least 5% greater, between 1% and 5% greater, 10% greater, between 5% and 10% greater, at least 15% greater, at least 20% greater, at least 25% greater, at least 30% greater, at least 40% greater, at least 50% greater, at least 75% greater, at least double the value, etc.), it can be determined that the user potentially has facial droop. In other examples, a first value for a first parameter is determined and a second value for a second parameter is determined, wherein the first value is compared with the second value to determine the presence of facial droop. A distance value for the left side of the face can be compared with a distance value for the right side of the face, wherein if one value exceeds the other beyond a predetermined threshold, it can be determined that the patient displays facial droop (e.g., on the side having a greater value, on the side having a smaller value, etc.). Additionally or alternatively, the parameter can be flagged if less than a threshold, within a threshold range, outside of a threshold range, or otherwise valued.

The set of anatomical features are preferably selected such that they include at least one feature along a midline of the patient's face and a pair of features which are normally (e.g., in the absence of droop) symmetric about this midline and which are in a region which typically shifts in the event of facial droop. This allows for any delta that occurs in the associated measurements of these features to be used to determine if facial droop has occurred and identify which side of the face is affected. Additionally or alternatively, features can be assessed and/or measured relative to a non-midline feature (e.g., relative to a true horizontal line, relative to a true vertical line, non-midline anatomical feature, etc.), relative to each other in any suitable way, or otherwise determined.

In a specific example, the visual assessment includes visually assessing a photo and/or a video of the patient for facial droop, wherein the visual assessment uses a set of computer vision processes to identify a set of landmarks on the patient's face, the landmarks including: each of the patient's eyes (e.g., center of pupils), the tip of the patient's nose, and each of the corners of the patient's mouth. Based on a set of distances between these landmarks (e.g., distance between each eye and the tip of the nose, distance between each corner of the mouth and the tip of the nose, a distance from a first eye to the tip of the nose to a first corner of the mouth for each side of the face, the difference in distance between a distance corresponding to the right side of the face and a distance corresponding to the left side of the face, etc.), a determination of facial droop can be made.

The process for determining facial droop is preferably robust with respect to tilting, twisting, rotating, turning, and/or otherwise manipulating the user's head. In the specific example shown in FIGS. 5A-5C, for instance, a lack of facial droop is determined in both the frames shown in FIGS. 5A and 5B, despite the patient reorienting his head.

Visually assessing the patient can additionally or alternatively include determining one or more parameters associated with a gaze of the patient, such as a gaze deviation (e.g., bias, Prevost's sign, etc.) toward a particular direction (e.g., left side, right side, up, down, etc.). A gaze parameter can include any or all of: a binary presence of gaze deviation, an amount of gaze deviation (e.g., angle with respect to forward), direction of gaze deviation (e.g., particular angle, left or right, etc.), duration of gaze deviation, or any other suitable parameter. Determining the gaze parameter preferably includes looking at one or both of the user's pupils (e.g., manually, with image processing of a photo, with image processing of a video or live stream, etc.) and determining whether or not they are centered. Additionally, determining the gaze parameter can include determining an amount (e.g., angle, distance, etc.) at which they are off center.

The gaze deviation can be determined based on a photo, set of photos, and/or a video, and is preferably determined with a set of trained neural nets, wherein the trained neural nets are trained to identify and track the pupils of the patient. The neural nets are further preferably finetuned based on images corresponding to stroke patients in order to accurately identify gaze deviation, but can additionally or alternatively be otherwise trained and/or finetuned. Further additionally or alternatively, gaze deviation can be determined in any other suitable way(s) (e.g., without machine learning).

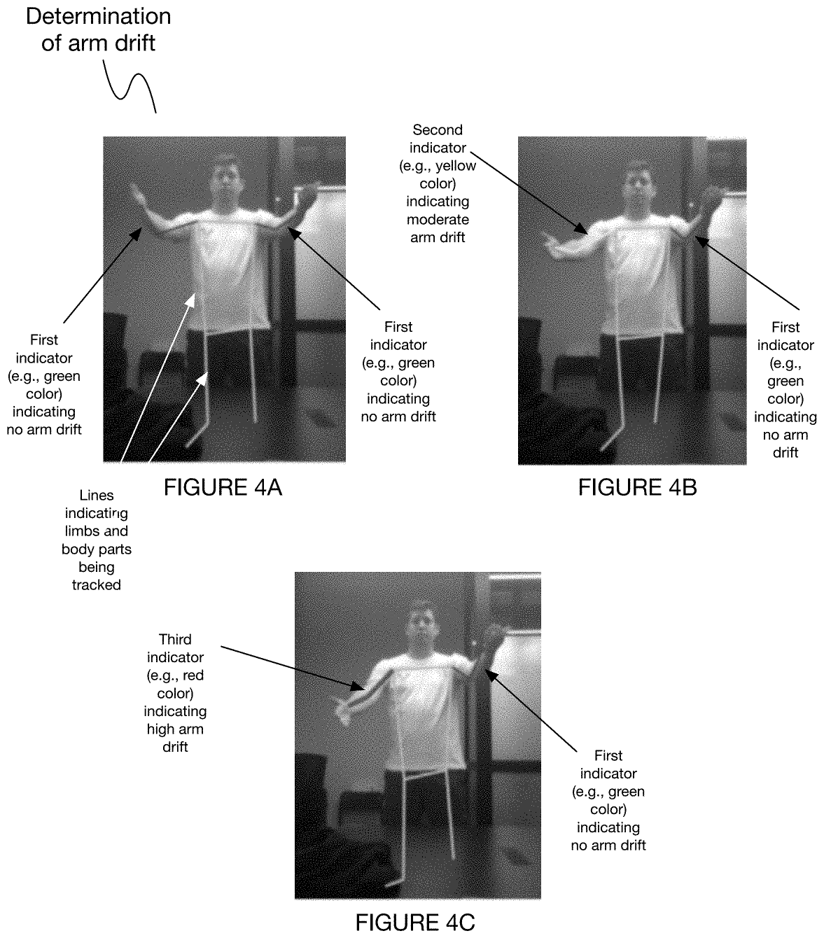

Visually assessing the patient can further additionally or alternatively include performing a body assessment of the user, which functions to determine one or more body-related stroke indicators, such as arm drift, wherein one or both arms drifts downward when held straight out (e.g., as shown in FIGS. 4A-4C). Determining arm drift can include instructing a patient to raise and/or lower one or both arms, and seeing if an arm drifts/drops (e.g., reaches a lower maximum height in comparison to the other arm, reaches a lower maximum height in comparison to an expected height, etc.). An arm drift parameter can additionally be determined (e.g., calculated), which can include any or all of: a binary presence of arm drift, an arm (e.g., right, left) experiencing drift, an amount of time required to raise one or both arms, an amount of time required to lower one or both arms, a rate at which an arm is raised, a rate at which an arm is lowered, an angle of bend in an arm, or any other suitable parameter.

In preferred variations, a set of trained neural nets (e.g., deep neural nets, convolutional neural nets, etc.) is used to identify (and optionally track in a video) the limbs (e.g., arms) of the user. The limbs are preferably identified and tracked based on a predetermined set of joints (e.g., wrists, shoulder joints, elbows, finger joints, etc.), but can additionally or alternatively be otherwise identified. The set of neural nets is preferably finetuned based on images and/or videos of stroke patients (e.g., to learn what limb drift looks like in stroke), but can additionally or alternatively be otherwise trained and/or finetuned. Additionally or alternatively, limb drift can be determined in absence of machine learning.

In the specific example shown in FIGS. 4A-4C, a video of the patient is taken and used to identify if and/or to what extent one or more arm drifts when asked to hold out his arms as shown in FIG. 4A. In FIG. 4A, no arm drift is initially detected, which can be indicated, for instance, in a first color (e.g., green) for the lines tracking both arms. In FIG. 4B, a moderate amount of drift (e.g., drift distance above a first threshold, drift above 5%, drift above 10%, drift above 15%, drift above 20%, drift above 25%, drift above 30%, drift greater than 1 centimeter [cm], drift greater than 5 cm, drift greater than 10 cm, drift greater than 20 cm, drift greater than 50 cm, etc.) is experienced by the user, which can be indicated, for instance by a second color (e.g., yellow) for the limb experiencing drift, which in FIG. 4B is the patient's right arm. In FIG. 4C, a high amount of drift (e.g., drift distance above a second threshold greater than the first threshold, drift above 5%, drift above 10%, drift above 15%, drift above 20%, drift above 25%, drift above 30%, drift above 50%, drift greater than 1 cm, drift greater than 5 cm, drift greater than 10 cm, drift greater than 20 cm, drift greater than 50 cm, etc.) is experienced by the user, which can be indicated, for instance by a third color (e.g., red) for the limb experiencing drift, which in FIG. 4C is the patient's right arm.

Additionally or alternatively, any other suitable parameters (e.g., gait parameters) can be determined during a visual assessment.

Any or all of the visual parameters can be determined based on image processing, manually inspected and recorded by a user (e.g., into the client application), prompted by any suitable trigger (e.g., prompting by a client application, part of a normally occurring emergency responder protocol, etc.), performed in absence of a prompt, or otherwise performed.

Assessing the patient S210 can additionally or alternatively include communicating with a specialist S214, which functions to supplement the assessment of the patient with an additional and expert opinion. The specialist is preferably associated with a potential point of care (e.g., hub), further preferably an advanced point of care (e.g., stroke center hub), such as an on-call neurospecialist (e.g., neurosurgeon, neurologist, etc.). In variations with a neurospecialist, the neurospecialist is preferably qualified to treat (e.g., operate on) stroke patients, but can additionally or alternatively be trained to assist in treatment (e.g., as a nurse, physician's assistant, etc.), be a non-physician (e.g., trained in the detection of stroke symptoms), and/or can be otherwise trained. In preferred variations, for instance, the specialist is associated with (e.g., currently employed, currently on duty, currently off duty, currently on-call, remote from, would accept treating the patient if the patient is transferred to a hub, etc.) a comprehensive stroke center, and can partake in any or all of: supplementing and/or wholly performing a patient assessment, recommending a point of care for the patient (e.g., the specialist's own facility for comprehensive stroke treatment, a non-specialist facility in determining that the symptoms are not life-threatening, etc.), agreeing to personally treat the patient, recommending that a colleague treat the patient (e.g., when the specialist on a video call is off duty), diagnosing a patient condition, speaking with the patient (e.g., to ask a set of questions, to further assess, etc.), or otherwise interacting with any of the users (e.g., emergency responder, patient, patient's family, driver, etc.) in making any suitable determination.

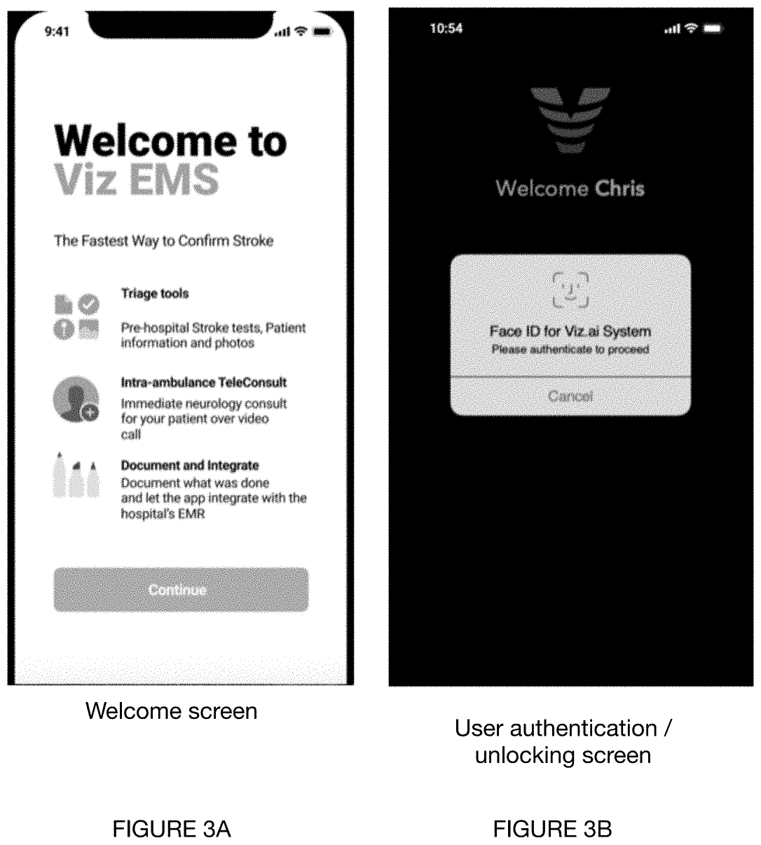

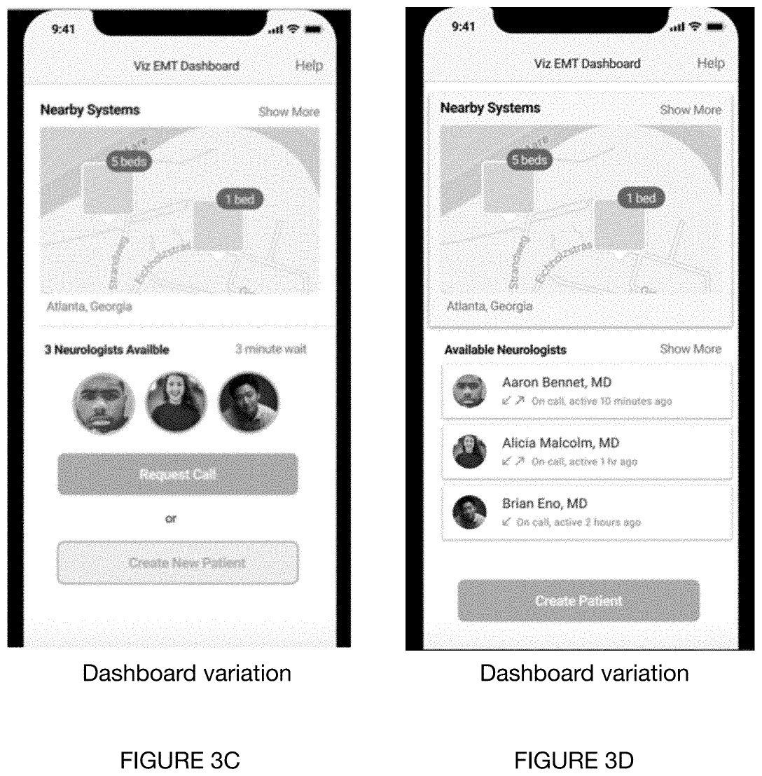

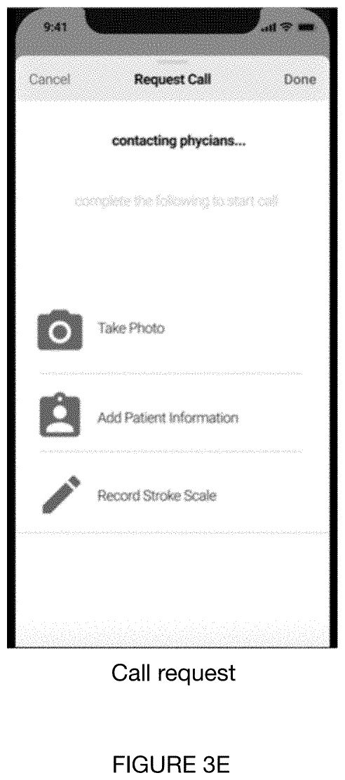

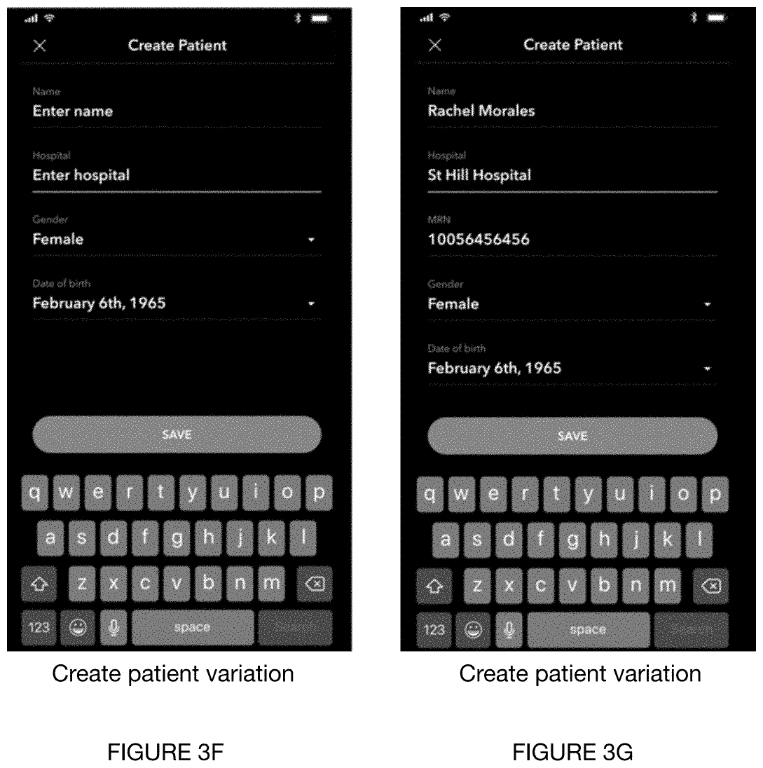

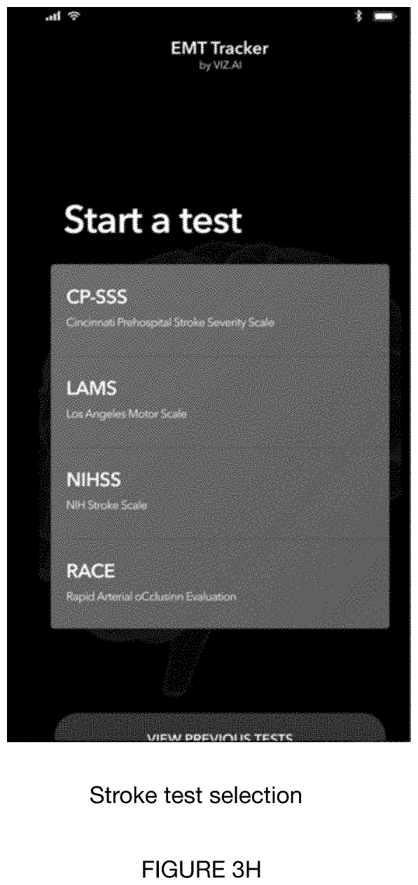

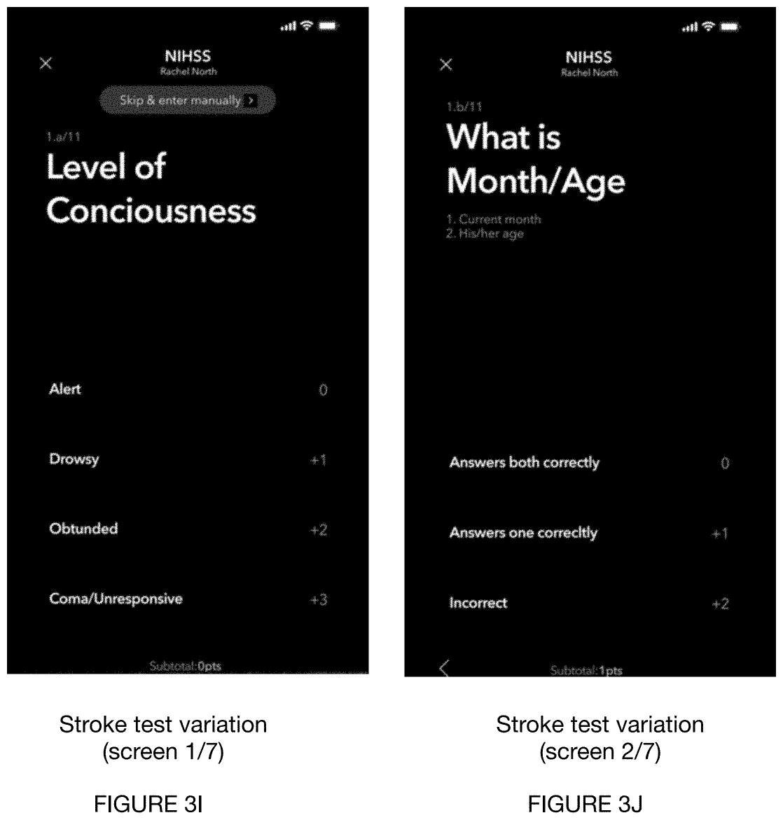

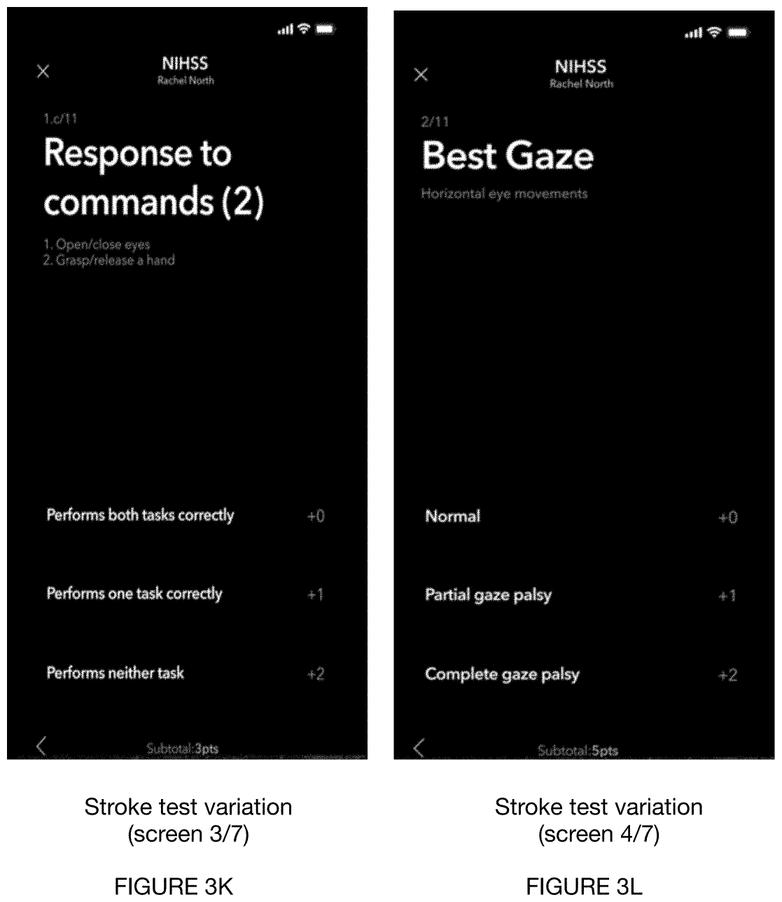

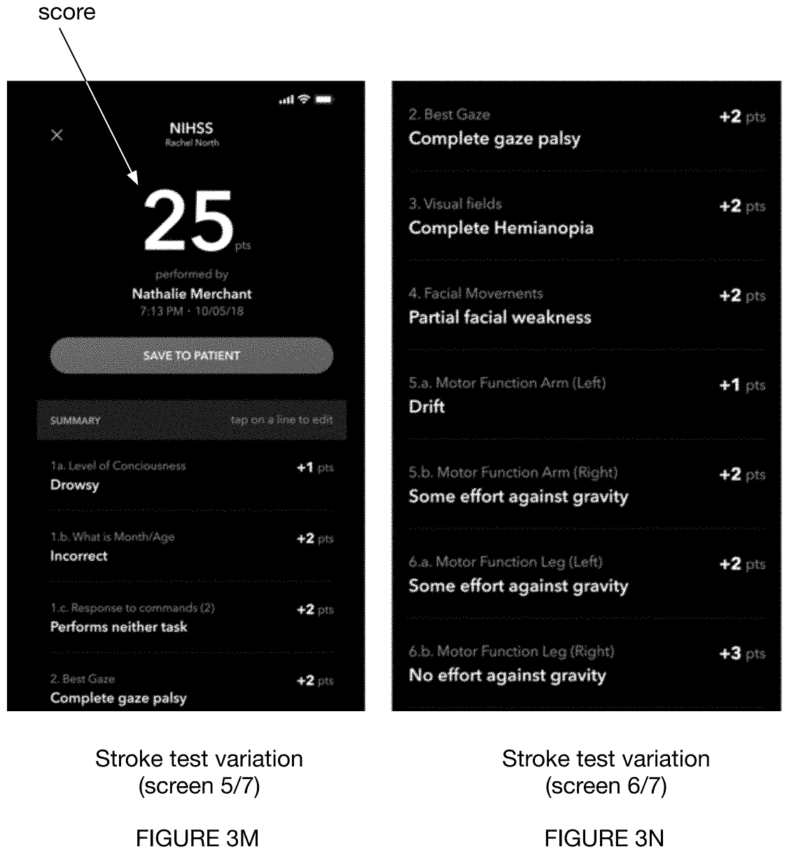

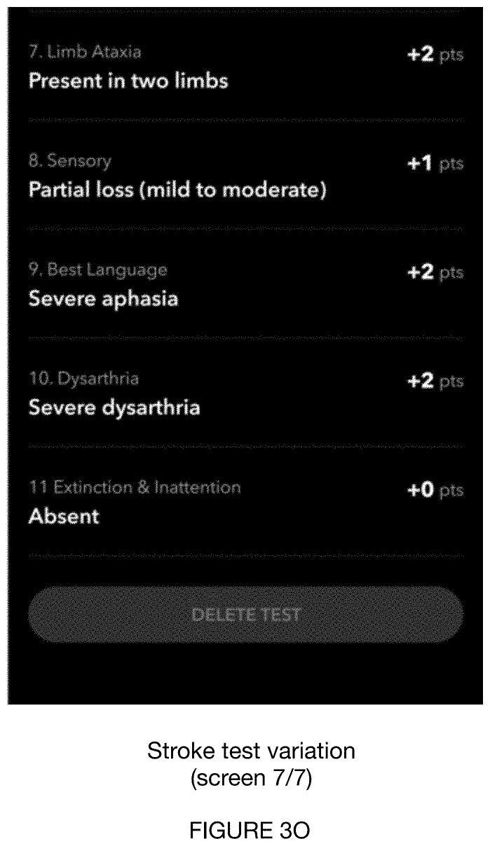

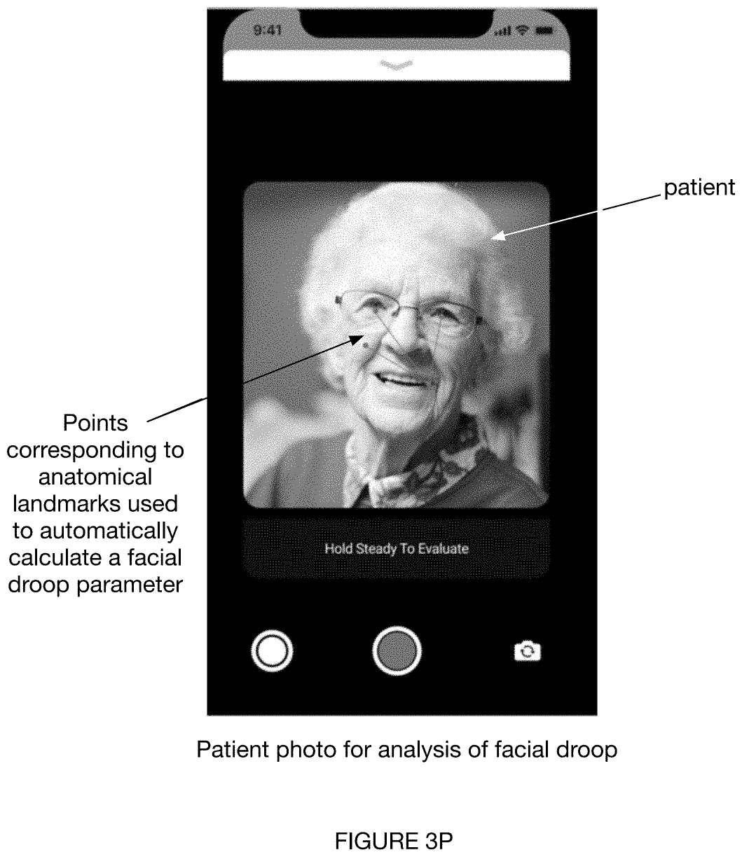

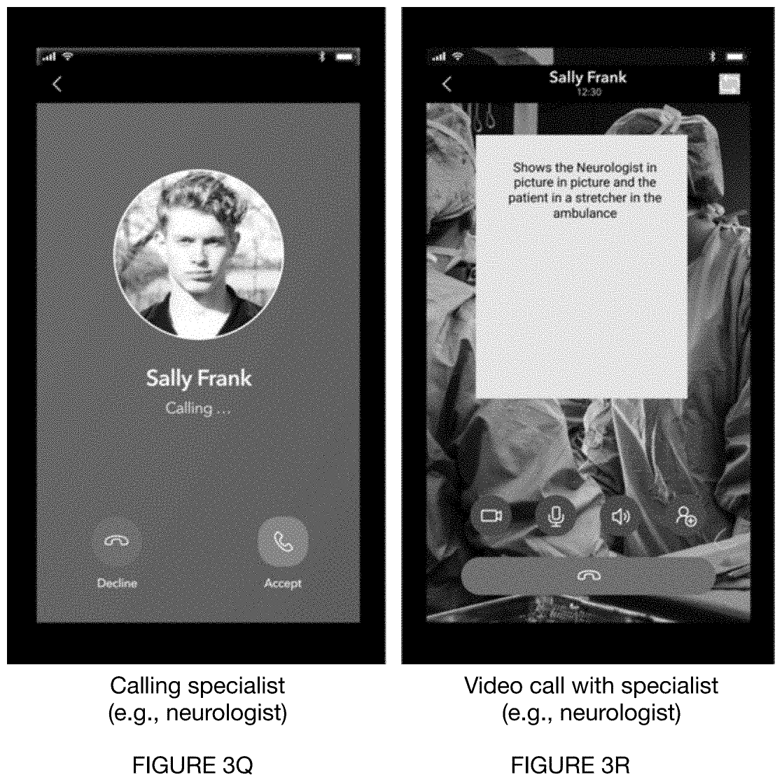

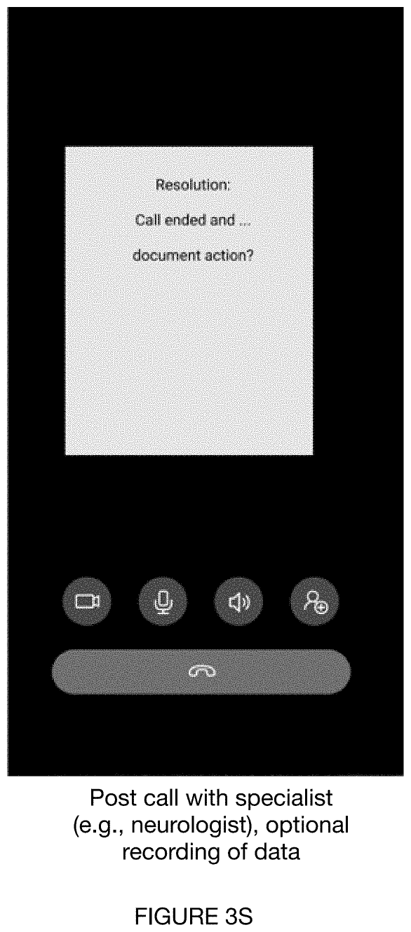

Initiating communication with the specialist is preferably triggered based on a selection of a specialist by an emergency responder at the client application (e.g., as shown in FIGS. 3C-3D). The specialist can be any or all of: selected from a set of multiple specialist options, automatically determined and optionally automatically called, selected from a prioritized list, self-selected and/or volunteered (e.g., wherein an initial ask for availability is sent to each of the specialist options and the first specialist to respond is selected as the specialist option, wherein an initial ask for availability is sent to each of the specialist options and specialists who do not respond are eliminated, etc.), and/or the specialists can be otherwise determined.

The specialist and/or specialist options can be determined based on any or all of: proximity of the specialist (e.g., to the patient's location, to an ambulance, to a point of care such as a stroke center, etc.), availability or predicted availability (e.g., based on last activity in client application, time since last active on the client application, specialist's amount of usage of the client application, etc.), specialist schedule (e.g., currently not scheduled for a procedure, appointment, surgery, call, etc.), expertise (e.g., in stroke, in a particular type of stroke, in a particular procedure, etc.), experience, and/or based on any other suitable factors. Any or all of these factors can optionally be provided to the emergency responder and/or to any other user at the client application. In specific examples (e.g., as shown in FIG. 3D), for instance, whether or not a specialist is on call and the time since they were last active on the client application is provided to the emergency responder at the client application.

Selection of the specialist preferably triggers a communication to be established between the emergency responder and the specialist. In preferred variations, for instance, the selection of the specialist establishes communication between a first client application executing on a first user device of the emergency responder and a second client application executing on a second user device of the specialist. The established communication is preferably established between the client applications, and can include any or all of: messaging (e.g., creating/initiating a messaging thread between the client applications), a call (e.g., initiating a call between the client applications and/or user devices), a video call (e.g., initiating a video call between the client applications and/or user devices), the sending/sharing of contact information between client applications and/or user devices, and/or any other suitable types of communication. Additionally or alternatively, any or all of the communication can be established at (e.g., call, trigger, initiate, etc.) secondary applications of the user devices (e.g., native text messaging applications, native call applications, etc.) and/or can use any other suitable applications and/or tools.

The method can optionally include selecting a second specialist (e.g., automatically selecting a second specialist, prompting the emergency responder to select a second specialist, etc.) in an event that the first specialist does not respond in a predetermined period of time (e.g., 30 seconds or less, 1 minute or less, 2 minutes or less, 3 minutes or less, 5 minutes or less, greater than 5 minutes, between 30 seconds and 2 minutes, etc.). The predetermined period of time can be measured from any or all of the following times: the time a first specialist is selected, the time a first specialist is called, the time since a stroke test is initiated, the time since a stroke score is calculated, and/or from any other suitable time(s).

Communicating with the specialist S214 preferably includes engaging in a video call (e.g., video conference call) with a specialist. The subject of the video call is preferably the patient, such that the specialist can visually assess the patient for one or more stroke signs, such as—but not limited to—those described above in performing one or more stroke tests S212 (e.g., wherein the video call is performed in addition to one or more stroke tests, wherein the video call replaces one or more stroke tests, etc.). As such, the video call can include the emergency responder (or any other user, the patient himself, etc.) pointing a camera of a user device (e.g., coupled to a client application) toward the patient (e.g., while the patient is in an ambulance, while the patient is partaking in a stationary assessment, etc.). Additionally or alternatively, communicating with the specialist S214 can include any or all of: a phone call with a specialist, messaging with a specialist (e.g., text messaging, messaging through a client application, etc.), transmitting data to a specialist (e.g., image data of the patient), or otherwise communicating with a specialist. Further additionally or alternatively, S214 can include the specialist accessing and/or reviewing data obtained during S210, such as data collected during S212. The data is preferably accessible through the specialist through a client application as described above (e.g., on a user device of the specialist, on a workstation of the specialist, etc.), but can additionally or alternatively be accessible to the specialist through a remote storage system, database, or any other information source.

S214 can optionally include sending a data packet (e.g., image of patient, video recording of patient, audio recording of patient, etc.) and an associated notification to the specialist, wherein the notification prompts the individual to review the data packet at the device (e.g., a message reciting “urgent: please review!”). Additionally or alternatively, the notification can prompt the individual to review data (e.g., original data packet, uncompressed images, etc.) at a separate device, such as a workstation in a healthcare facility, a PACS server, or any other location. Further additionally or alternatively, the notification can include any suitable information, such as, but not limited to: instructions (e.g., for treating patient, directions for reaching a healthcare facility, instructions determined based on one or more stroke tests, etc.), contact information (e.g., for emergency physician at first point of care, administrative assistant, etc.), patient information (e.g., patient history), or any other suitable information.

The notification preferably includes an SMS text message but can additionally or alternatively include an email message, audio message (e.g., recording sent to mobile phone), push notification, phone call, or any other suitable notification.

The information is preferably sent to the device through a client application executing on the user device but can additionally or alternatively be sent through a messaging platform, web browser, or other platform.

In some variations, a notification is sent which prompts the individual to provide an input, wherein the input can indicate that the individual will view, has viewed, or is in the process of viewing the information (e.g., image data, stroke test data, etc.), sees the presence of a condition (e.g., true positive, serious condition, time-sensitive condition, etc.), does not see the presence of a condition (e.g., false positive, serious condition, time-sensitive condition, etc.), has accepted treatment of the patient (e.g., swipes right, swipes up, clicks a check mark, etc.), has denied treatment of the patient (e.g., swipes left, swipes down, clicks an ‘x’, etc.), wants to communicate with another individual (e.g., healthcare worker at 1^{st} point of care), such as through a messaging platform (e.g., native to the device, enabled by the client application, etc.), or any other input. In some variations, one or more additional notifications are provided to the individual (e.g., based on the contents of the input), which can be determined by a lookup table, operator, individual, decision engine, or other tool. In one example, for instance, if the individual indicates that the condition is a true positive, information related to the transfer of the patient (e.g., estimated time of arrival, directions to the location of the patient, etc.) can be provided (e.g., in a transfer request, wherein patient transfer to a specified location, such as the 2^{nd} point of care, can be initiated upon transfer request receipt). In some variants, the data (e.g., images) are displayed on the user device (e.g., mobile device, workstation) in response to user interaction with the notification (e.g., in response to input receipt). However, the input can trigger any suitable action or be otherwise used.

S214 and/or any other processes of the method 200 can additionally or alternatively include triggering, enabling, and/or initiating communication between any suitable users. The communication is preferably established at and/or triggered by the client application, but can additionally or alternatively be established at and/or triggered by any other suitable tools, components, and/or systems (e.g., paging system of healthcare facility, workstation of healthcare facility, etc.). Communication can be established, for instance, between any or all of: multiple specialists (e.g., the specialist involved in the teleconsultation and the specialist treating and/or eventually treating the patient) and/or physicians (e.g., specialist on teleconsultation and members of patient's care team; between multiple members of patient's care team; etc.); between a specialist and a clinical trial research coordinator; between an emergency responder and a clinical trial research coordinators; and/or between any other suitable users. This communication and/or any other communication can be triggered at any point during, prior to, and/or after the method, such as: in response to a teleconsultation (equivalently referred to herein as a teleconsult) being performed, in response to a stroke score being calculated, in response to a specialist being assigned to the patient for treatment, in response to the patient reaching a point of care, and/or at any other suitable times.

Additionally or alternatively, S214 and/or any other processes of the method can include and/or trigger the integration of the client application with a healthcare facility database (e.g., EMR database, EHR database, PACS, etc.), such that information can be shared from the client application to the database and/or from the database to the client application. The healthcare facility is preferably the point of care to which the patient is traveling and/or located at, but can additionally or alternatively include any other suitable healthcare facility.

S214 can be performed during S212, prior to S212, after S212, in absence of S212, or in any combination of these times. Alternatively, S210 can be performed in absence of S214.

In some variations, S214 is triggered based on the results of S212. In an example, for instance, a patient score based on the assessment in S212 is calculated, which indicates a potential stroke. This can subsequently trigger a call or other notification (e.g., text message, message through a client application, etc.) to a specialist (e.g., neurospecialist) to confirm. Additionally or alternatively, S214 can be performed based on a trigger indicating that S212 has not been performed to a desired degree (e.g., not initiated, only partially performed, etc.) and/or within a predetermined time period. Further additionally or alternatively, S212 and S214 can always be performed during the method, only performed in absence of the other, initiated by user selection (e.g., decided by emergency responder), or otherwise performed.

Additionally or alternatively, S214 can require that S212 be performed in order to contact a specialist in S214. In some variations (e.g., as shown in FIG. 3E), completion of any or all of the following is suggested, required, and/or checked for prior to initiating a call or other form of contact to the specialist: taking a photo of the patient, recording patient information (e.g., demographic information, list of symptoms, etc.), recording a score from a stroke test (e.g., stroke scale).

S210 can optionally including determining a patient condition S216, which can function to inform any or all of the following processes of the method (e.g., match the patient with a point of care, The patient condition can be a suspected/predicted patient condition, a confirmed patient condition (e.g., by a specialist), and/or any other suitable patient condition. The patient condition can be determined based on any or all of the information and/or processes described above (e.g., aggregated based on stroke tests and image processing); the specialist opinion; a set of algorithms and/or machine learning processes; and/or based on any other suitable information.

Alternatively, the method 200 can be performed in absence of S216; S216 can be performed later in the method 200; and/or the method 200 can be performed in any other suitable way.

In one example, S210 includes prompting an emergency responder (e.g., through a client application) to initiate a video call with a specialist after a predetermined time since reaching the patient has passed in the absence of performing one or more stroke tests (e.g., through the client application). In another example, S210 includes an emergency responder deciding to initiate a video call with a neurospecialist after suspecting that the patient may potentially be displaying one or more indicators of stroke.

S210 can optionally include determining (e.g., calculating) a patient score based on any or all of: S212, S214, and any other process for assessing the patient. The patient score preferably indicates a likelihood (e.g., percentage likelihood, score along a predetermined scale, etc.) that the patient is experiencing a stroke. The score can be at least partially automatically determined (e.g., at the client application based on information entered into the client application, etc.), determined by a user (e.g., qualitatively assigned by an emergency responder, qualitatively assigned by a specialist, verified and/or modified by a specialist, etc.), predicted using one or more deep learning algorithms, or otherwise determined. The score can be a conventional stroke score (e.g., associated with predetermined stroke tests as described above), a customized stroke score (e.g., based on the client application), or any other suitable score or set of scores.

In some examples, the patient score is determined automatically at the client application based on the inputs to one or more stroke tests which are recorded at the client application. In some examples, the patient score is used as a trigger for S214 (e.g., contact specialist if score is above a predetermined threshold, contact specialist if score is below a predetermined threshold, etc.). Additionally or alternatively, the score can be used to determine a point of care (e.g., hub facility if above a predetermined threshold, spoke facility if below a predetermined threshold, etc.), diagnose a patient condition, be recorded and provided for a physician to use when assessing the patient after the patient reaches the point of care of the physician, or otherwise used.

### 4.3 Method—Triaging the Patient Based on the Assessment S250

The method can include triaging the patient based on the assessment S250, which functions to transfer the patient to a point of care for treatment (e.g., nearest stroke center in the case of a suspected stroke, nearest facility in the case of a suspected less serious condition, etc.). Additionally or alternatively, S250 can function to diagnose the patient; recommend a treatment option (e.g., to a specialist) for the patient; assign the patient to a particular specialist, care team, or healthcare facility group; schedule a procedure and/or treatment for the patient at a point of care; or otherwise recommend and/or initiate a next step for the patient. Triaging the patient is preferably determined based on results of S210 (e.g., based on a patient score, based on a specialist decision, etc.), but can additionally or alternatively be based on any suitable information.

An output of S250 preferably includes a point of care for the patient to be transferred to (e.g., from an ambulance, from a first point of care, etc.) and subsequently treated at. Additionally or alternatively, S250 can produce any other suitable output, such as—but not limited to—any or all of: a particular specialist, a prescribed treatment option (e.g., surgery, operation, prescribed stay at the facility, rehabilitation plan, drug, etc.), a clinical trial for which the patient would be a candidate, or any other suitable outcome.

In some variations, information stored and/or referenced in a lookup table (e.g., stored at a user device, stored at a client application, accessible by a client application, in remote storage, etc.) which associates the information with one or more points of care, a specialist, treatment option, or any other suitable information, can be used in triaging the patient.

The lookup table is preferably determined based on multiple types of information, such as, but not limited to: location information (e.g., location of a 1^{st} point of care, location of a 2^{nd} point of care, distance between points of care, location of a point of care relative to real time or near real time location of the ambulance, location of an ambulance relative to a point of care, GPS coordinates of a hub facility and GPS coordinates of a spoke facility, GPS coordinates of an ambulance, etc.), temporal information (e.g., time of transit between points of care, time passed since patient presented at 1^{st} point of care, etc.), features of condition (e.g., size of occlusion, severity of condition, etc.), patient demographics (e.g., age, general health, history, etc.), specialist information (e.g., schedule, on-call times, historic response time, skill level, years of experience, specialty procedures, historic success or procedures, etc.), healthcare facility information (e.g., current number of patients, available beds, available machines, etc.), but can additionally or alternatively be determined based on a single type of information or in any other suitable way. Information can be actual, estimated, predicted, or otherwise determined or collected.

S250 can optionally include enrolling a patient in a clinical trial or recommending the patient for enrollment in a clinical trial. Matching with a clinical trial can be performed based on any or all of: a stroke test score, a video call with a specialist, a video call with a research coordinator, processing of images and/or video in a visual assessment, and/or any other suitable information.

In some variations, S250 includes transferring the patient to a comprehensive stroke center based on a patient score determined from a series of stroke tests performed while the patient is in an ambulance and/or at the location at which the ambulance picks up the patient, wherein the patient score exceeds a predetermined threshold.

4.4 Method—Collecting Data Associated with the Patient S260

The method 200 can optionally include collecting data associated with the patient S260, which can function make data accessible for present or later use (e.g., for a specialist to assess when the patient reaches the specialist's point of care, for a specialist to assess while the patient is in transit, etc.), be integrated into a patient's health record (e.g., EMR database, EHR database, PACS, etc.), be aggregated with other data of the patient and/or with other patient data (e.g., to be used in training a deep learning algorithm, to be used in a clinical trial, etc.), or otherwise used.

The method can additionally or alternatively include initiating the transfer of a patient, wherein the transfer includes a recommendation that the patient be considered for a clinical and/or research trial, based on one or more of: a suspected clinical condition of the patient (e.g., ICH), patient information (e.g., demographic information), a patient's willingness or potential willingness to participate, and/or any other suitable information. Initiating the recommendation can include transmitting any or all of the notifications described above (e.g., text message, call, email, etc.) to a specialist involved in the clinical and/or research trial, a specialist who has actively turned on notifications for clinical trial recruitment, a researcher, a research principal investigator, an administrative assistant, the patient himself, or any other suitable entity or individual.

### 4.8 Method—Aggregating Data S260

The method 200 can optionally include any number of sub-steps involving the aggregation of data involved in and/or generated during the method 200, which can function to improve future iterations of the method 200 (e.g., better match patients with a specialist, decrease time to treat a patient, increase sensitivity, increase specificity, etc.). The aggregated data is preferably used in one or more analytics steps (e.g., to refine a treatment option, make a recommendation for a drug or procedure, etc.), but can additionally or alternatively be used for any other suitable purpose.

In some variations, for instance the outcomes of the patients examined during the method 200 are recorded and correlated with their corresponding data packets, which can be used to assess the success of the particular treatment options chosen and better inform treatment options in future cases.

### 4.9 Method—Variations

In one variation of the method 200 (e.g., as shown in FIG. 1), the method includes: initiating an assessment S205 (e.g., verifying an identity of an emergency responder interacting with a client application of a first user device); assessing the patient S210 which can include any or all of: administering a stroke test and/or stroke scale (e.g., Cincinatti Prehospital Stroke Severity Scale [CP-SSS], Los Angeles Motor Scale [LAMS], National Institute of Health Stroke Scale [NIHSS], Rapid Arterial oCclusion Evaluation [RACE], etc.), communicating with a specialist (e.g., video tele-consult with a neurologist, transmitting a patient photo to a neurologist, etc.), and visually assessing the patient (e.g., automatically determining a facial droop parameter based on a photo of the user); determining a patient condition S216 (e.g., suspected stroke based on a patient stroke score determined from assessing the patient); triaging the patient based on the assessment S250 (e.g., to a comprehensive stroke center if a calculated stroke score is above a predetermined threshold and to a general ER if the score is below a predetermined threshold); and collecting and integrating the assessment data S260 (e.g., at a patient profile, at a patient database, etc.).

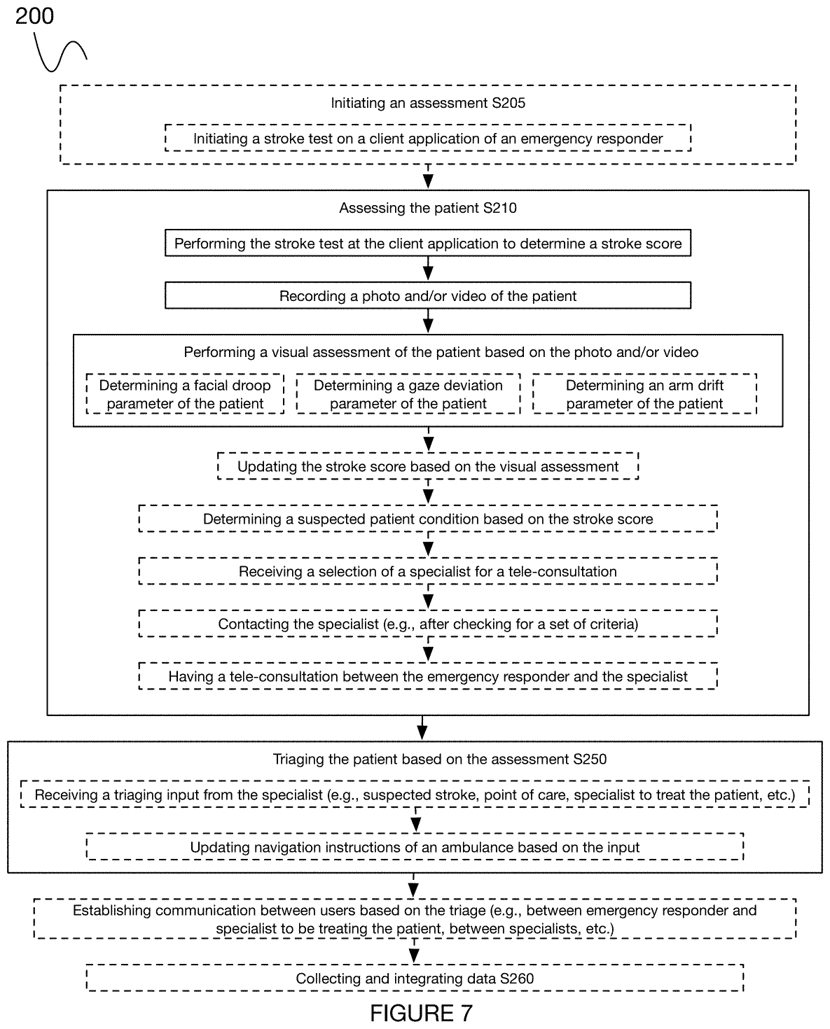

In a specific example (e.g., as shown in FIG. 7), the method 200 includes any or all of: initiating a stroke test on a client application of an emergency responder; performing the stroke test at the client application to determine a stroke score; recording a photo and/or video of the patient; performing a visual assessment of the patient based on the photo and/or video, which can include any or all of determining a facial droop parameter of the patient, determining a gaze deviation parameter of the patient, determining an arm drift parameter of the patient, and/or any other suitable assessments; updating the score based on the visual assessment; determining a suspected patient condition based on the stroke score; receiving a selection of a specialist for a tele-consultation; contacting the specialist; having a tele-consultation between the emergency responder and the specialist; receiving a triaging input from the specialist (e.g., point of care to take the patient, specialist to treat the patient, suspected stroke confirmation, etc.); updating navigation instructions of an ambulance based on the triaging input; establishing communication between users based on the triage (e.g., between emergency responder and specialist to be treating the patient, between specialists, etc.); collecting and integrating data; and/or any other suitable processes. The processes in this example and in any variation or example of the method can be performed in any combination and/or any order.

In a specific example (e.g., as shown in FIGS. 3A-3S) of a client application 120 used in this variation, assessing the patient through the client application includes any or all of: receiving an input at the client application from an emergency responder (e.g., to initiate a triage tool such as a pre-hospital stroke test, to initiate an intra-ambulance tele-consult with a specialist, to document and integrate patient information, etc.); verifying an identity of the user, such as an emergency responder (e.g., through face identification); displaying a map of point of care locations (e.g., and bed availability at each location); displaying a set of neurologist options (e.g., available neurologists) for a tele-consult; receiving an input from the user (e.g., to request a call with a neurologist, to create a new patient profile for assessment, etc.); in the event that the user requests a call (e.g., as shown in FIG. 3E), contacting the specialist and providing a set of inputs for the user to optionally enter patient information (e.g., a user photo, demographic information, stroke test information, observations, etc.); in the event that the user creates a new patient profile (e.g., as shown in FIG. 3F, as shown in FIG. 3G, etc.), providing a set of inputs for the user to enter patient demographic information; receiving, at the client application, a selection from the user for a particular stroke test to perform; providing a set of prompts and/or commands to be provided to the patient for the stroke test, and receiving the response of the patient; calculating a score based on the patient's performance of the stroke test; analyzing a photo of the patient (e.g., with computer vision) to determine if the patient is experiencing facial droop; engaging in a video call with a specialist; and any other suitable processes. The processes described above can be performed in any suitable combination and order.

Additionally or alternatively, the method can include any other steps performed in any suitable order.

Although omitted for conciseness, the preferred embodiments include every combination and permutation of the various system components and the various method processes, wherein the method processes can be performed in any suitable order, sequentially or concurrently.

As a person skilled in the art will recognize from the previous detailed description and from the figures and claims, modifications and changes can be made to the preferred embodiments of the invention without departing from the scope of this invention defined in the following claims.

We claim:

1. A method for mobile triage of a patient in an ambulance, the method comprising:
receiving, at a first client application executing on a first user device, the first user device associated with an emergency responder, a request to initiate a stroke test at the first client application;
at the first client application, initiating the stroke test, wherein the stroke test comprises receiving, at the first client application, an entry of at least the following:
a level of consciousness of the patient;
a rating of a set of patient answers to a set of questions;
a rating of a motor function of the patient;
automatically calculating a stroke score based on the stroke test;
receiving, at the first client application, patient demographic information;
recording, at the first user device, at least one of a photo and a video of the patient;
at a remote computing system, determining, based on the at least one of a photo and a video of the patient, at least one of:
a facial droop parameter;
a gaze deviation parameter; and
an arm drift parameter;
at the first client application:
displaying a set of specialist options;
receiving a selection of a selected specialist from the set of specialist options, wherein the selected specialist is associated with a second user device executing a second client application;
requesting a video call with the selected specialist;
in response to the selected specialist accepting the video call, establishing a communication between the first and second client applications; and
at the first client application, updating a point of care of the patient based on an input from the selected specialist.

2. The method of claim 1, further comprising, in response to the stroke score exceeding a predetermined threshold, prompting the selection of the selected specialist at the first client application.

3. The method of claim 1, further comprising checking for a set of criteria to be completed prior to initiating the video call with the selected specialist, wherein the set of criteria comprises:
an entry of the patient demographic information;
the stroke score; and
the at least one of:
the facial droop parameter;
the gaze deviation parameter; and
the arm drift parameter.

4. The method of claim 1, further comprising displaying, at the first client application, a map including a set of points of care proximal to a current location of the ambulance.

5. The method of claim 4, further comprising displaying at the map, a number of beds available at each of the points of care.

6. The method of claim 1, wherein the stroke score is calculated at a computing subsystem of the first user device.

7. The method of claim 1, further comprising displaying, at the first client application, an availability of each of the set of specialists.

8. The method of claim 7, further comprising displaying, at the first client application, a time since active for each of the set of specialists, wherein the time since active for the selected specialist quantifies a time since the selected specialist was active on the second client application.

9. The method of claim 1, wherein, in an event that the specialist does not answer the call in a predetermined amount of time, a second specialist is automatically selected and called.

10. The method of claim 9, wherein the predetermined amount of time is 2 minutes or less.

11. The method of claim 1, wherein the second client application is configured to display the stroke test, the stroke score, the patient demographic information, and the at least one of a photo and a video to the selected specialist.

12. The method of claim 1, further comprising updating the point of care of the patient based on the call.

13. The method of claim 1, further comprising, in response to updating the point of care, establishing a communication between the first client application and a third client application, the third client application executing on a third user device associated with a second specialist, wherein the second specialist is located at the point of care and part of the patient's care team.

14. The method of claim 1, further comprising, in response to updating the point of care, establishing a communication between the second client application and a third client application, the third client application executing on a third user device associated with a second specialist, wherein the second specialist is located at the point of care and part of the patient's care team.

15. The method of claim 1, further comprising updating a set of navigation instructions of the ambulance based on the updated point of care.

16. The method of claim 1, wherein the communication comprises a messaging thread.

17. A method for mobile triage of a patient, the method comprising:
receiving, at a first client application executing on a first user device, the first user device associated with an emergency responder, a request to initiate a stroke test at the first client application;
at the first client application, initiating the stroke test;
automatically calculating a stroke score based on the stroke test;
receiving, at the first client application, patient demographic information;
recording, at the first user device, at least one of a photo and a video of the patient;
determining, based on the at least one of a photo and a video of the patient, a stroke parameter associated with the patient;
at the first client application:
displaying a set of specialist options;
receiving a selection of a selected specialist from the set of specialist options, wherein the selected specialist is associated with a second user device executing a second client application;
requesting a communication with the selected specialist at the second client application;
in response to the selected specialist accepting a request for communication, establishing the communication between the first and second client applications; and
at the first client application, updating a point of care of the patient based on an input from the selected specialist.

18. The method of claim 17, wherein the stroke test comprises receiving, at the first client application, an entry of at least the following:
a level of consciousness of the patient;
a rating of a set of patient answers to a set of questions; and
a rating of a motor function of the patient.

19. The method of claim 17, wherein the stroke parameter comprises at least one of:
a facial droop parameter;
a gaze deviation parameter; and
an arm drift parameter.

20. The method of claim 17, wherein the communication comprises a video call.

21. The method of claim 17, wherein the stroke score is automatically calculated at a remote computing system and wherein the stroke parameter is automatically determined based on a set of machine learning processes at the remote computing system.
